(19) United States
(12) Patent Application Publication
(10) Pub. No.: US 2008/0064038 A1
(43) Pub. Date: Mar. 13, 2008

(54) Autism Genes and Regulated Secretion

(75) Inventors: Jean-Pierre Fryns, Kessel-Lo (BE); Koenraad Devriendt, Holsbeek (BE); Willem Van De Ven, Leuven (BE); Jean Steyaert, Kessel-Lo (BE)

Correspondence Address: CLARK & ELBING LLP, 101 FEDERAL STREET, BOSTON, MA, 02110, US

(73) Assignee: K.U.LEUVEN RESEARCH AND DEVELOPMENT, Leuven (BE)

(21) Appl. No.: 11/720,056

(22) PCT Filed: Nov. 23, 2005
(86) PCT No.: PCT/BE05/00170

§ 371 Date: Aug. 20, 2007

(30) Foreign Application Priority Data
Nov. 23, 2004 (GB) 0425773.9
Dec. 9, 2004 (GB) 0427002.1

(51) Int. Cl. C12Q 1/68

(52) U.S. Cl. 435/6

(57) ABSTRACT

The present invention relates to genes encoding proteins involved in regulated secretion which are linked with the occurrence of or the susceptibility to a neural system disorder. The invention thus also relates to methods of identifying patients which have been diagnosed with a neural system disorder as susceptible to the treatment with modulators of regulated secretion.

### FIELD OF THE INVENTION

The present invention relates to screening methods for use in therapeutic diagnostics and genetic diagnostics of neural system disorders. The invention further relates to genetic alterations associated with neural system disorders and brain anomalies such as autism their onset and development and also to the encoded proteins of said genes associated with neural system disorders. The invention further relates to screening methods for therapeutics for the treatment and/or prevention of neural system disorders.

### BACKGROUND OF THE INVENTION

Autism is a severe developmental disorder of the central nervous system characterised by the clinical triad of abnormal language development, disturbance of social skills and particular behavioural features. The disorder starts at a young age, has a variable severity and additional medical problems often appear such as mental retardation (75%) or epilepsy (15%). The prevalence of autism is estimated at about 1/1000 to 1/2000. Because of its high prevalence and the need for a lifelong medical and pedagogical supervision, autism is a major burden not only for the families involved but also for public health in general. In 5-10% of the cases, autism is a symptom of a recognisable disorder but in most cases, the cause of autism is not known, and then called “idiopathic autism”.

Pathogenesis of autism involves a variety of structural brain anomalies, which have been identified in Magnetic Resonance Imaging (MRI) or post-mortem studies. The most consistent neuropathological findings in autism up to date, are abnormalities in the cerebellum. More specifically, a decreased number of Purkinje cells were found in 21 of all 23 reported postmortem cases. It is now clear that the cerebellum has an important role in diverse higher cognitive functions, such as the language and emotional control, besides its role in motor control. For these reasons, autism research has recently focused more on the cerebellum. Postmortem studies have implicated the glutamate neurotransmitter system in autism, and reduced levels of the anti-apoptotic protein bcl2 were demonstrated. Nevertheless, a single coherent theory explaining the pathogenesis of autism is lacking.

Family and twin studies have revealed that autism has a genetic origin, inherited as a polygenic disorder, with an estimated 2 to 10 interacting loci. The identification of the genes involved in the origin of autism is an appealing way to gain further insight. At present no genes for idiopathic autism are known. Results of association studies with candidate genes did not yield consistent results. Eight large genome screens failed to define small chromosomal regions harbouring susceptibility loci for autism, but several suggestive regions have emerged. Hence, there is a need for new ways to screen for autism. Positional cloning through chromosomal aberrations associated with autism is an alternative means to identify genes involved in autism.

Diagnosis of autism presents difficulties in its own right, and a number of modalities have been proposed primarily based upon psychiatric evaluations. A number of different therapies have been attempted in an effort to cure autism or at least lessen the clinical symptoms thereof. Such have included drug therapies as well as psychiatric care and attempted counselling. In general, results of such treatments have been disappointing, and autism remains very difficult to effectively treat, particularly in severe cases. The present invention thus aids in fulfilling these needs in the art.

The compartmentalization of biological processes into distinct membrane-bound organelles with unique protein and lipid composition is a central characteristic of eukaryotic cells. Therefore, transport of biomolecules from their site of synthesis or uptake to specific destinations has to be tightly controlled (Epstein F H. The New England Journal of Medicine 2000;343(15):1095-1104). The intracellular transport of proteins and lipids relies to a large extent on their sorting into specific vesicle populations, the directional movement of the vesicles through the cell, and the subsequent fusion of the vesicles with specific cellular compartments (Salaun C et al. Traffic 2004;5(4):255-64). The processes responsible for the correct localization of molecules within the cell are grouped under the term ‘membrane or vesicle trafficking’.

The Golgi complex functions to covalently modify newly synthesized proteins and lipids, and to sort and package these molecules into vesicles/granules for further transport to their site(s) of function (Mogelsvang S, et al. Traffic 2004;5(5):338-45). Two types of secretory pathways have been described, the constitutive and the regulated pathway. Both constitutive and regulated secretory vesicles are derived from the trans-Golgi network (TGN), the last station of the Golgi complex (Liu Y, Krantz D E, Waites C, et al. Trends Cell Biol 1999;9(9):356-63). The eventual fusion of these vesicles with the plasmamembrane (PM) occurs in a process called exocytosis.

Two types of secretory vesicles co-exist in the regulated secretory pathway, large dense-core vesicles (LDCVs; 70-100 nm in diameter (Liu et al, 1999, above)) and small synaptic vesicles (SSVs; ˜40 nm in diameter (Chuang J Z et al. J Neurosci 1999;19(8):2919-28). Neurons contain both SSV and LDCV populations of vesicles, whereas neuroendocrine cells contain dense-core secretory granules (counterparts of LDCVs), but not SSVs (Liu et al, 1999, above).

In general, LDCVs are formed at the TGN as immature secretory granules, which undergo maturation (processing and condensation of the cargo) during transport, resulting in a mature secretory granule (Thiele C, Huttner W B. Semin Cell Dev Biol 1998;9(5):511-6). In neurons, these vesicles are assembled at the soma and transported via axons to nerve endings (Chuang et al. 1999, above). After the release of vesicle content (Liu et al, 1999, above), LDCVs move by retrograde transport to the soma for reloading endings (Chuang et al. 1999, above).

On the other hand, SSVs are formed at the axonal endosomes and transported to an active zone at the nerve terminal (Liu et al, 1999, above). These vesicles mediate the rapid release required for information processing by the nervous system (Liu et al, 1999, above). Finally, the SSVs are recycled by endocytosis at the terminal endings (Chuang et al. 1999, above).

These vesicle populations also differ in their contents. SSVs contain classical transmitters, such as acetylcholine, GABA, serotonin and glutamate, whereas LDCVs contain neuropeptides, such as opioids (Liu et al, 1999, above). Moreover, monoamines differ from other classical transmitters and are released from both type of vesicles (Liu et al, 1999, above).

### SUMMARY OF THE INVENTION

The present invention relates to methods and tools for use in genetic and therapeutic diagnosis.

According to a first aspect of the invention, a method is provided for identifying a patient which has been diagnosed with a neural system disorder, more particularly an autism spectrum disorder, as susceptible to the treatment with a medicament capable of modulating targeted secretion, said method comprising detecting, in a biological sample of said patient, aberrant expression of one or more genes encoding proteins involved in regulated secretion.

A specific embodiment of this aspect of the invention relates to methods for identifying such patient groups described above, which comprise detecting aberrant regulated secretion, more particularly increased regulated secretion in cells, more particularly isolated cells, of said patient. These cells can be haematopoietic cells such as blood cells, including platelets, such as for instance obtained from a blood sample. Alternatively the cells can be other cells which have regulated secretion such as neural cells.

According to another embodiment, the screening method of the invention comprises detecting, at the DNA, RNA or protein level aberrant expression of one or more genes involved in regulated secretion. Such detection of aberrant expression comprises both a quantitative determination of expression levels as well as the identification of aberrant gene products, in a biological sample of the animal or human. More specifically such a method comprises the detection of one or more alterations, which are chromosomal or sequence alterations in the one or more genes involved in regulated secretion. Particular embodiments of the method of the invention relate to the detection of a functional alteration selected from the group consisting of a translocation, an inversion, a deletion, an insertion or a substitution.

In a particular embodiment such functional alterations detected in the one or more genes encoding proteins involved in regulated secretion result in a reduction or loss of function of said one or more genes.

A further aspect of the present invention provides a method of testing or screening an animal for a neural system disorder, more particularly an autistic spectrum disorder or a predisposition thereto which method comprises detecting, for at least two genes involved in regulated secretion, whether there is aberrant expression; whereby aberrant expression of at least one of these two genes is indicative of a neural system disorder, more particularly an autism spectrum disorder or a predisposition thereto. Specific embodiments of this aspect of the invention relate to methods which comprise detecting, at the DNA, RNA or protein level, aberrant expression levels of said genes and/or the expression of aberrant gene products in a biological sample of an animal, more particularly a human. More specifically, methods which rely on detection of altered mRNA transcripts or mRNA precursor levels are envisaged. Alternatively detection of specific alterations which rely on amplification of chromosomal material and identification by specific probes or by sequencing are envisaged. Further specific embodiments relate to methods which comprise the identification of one or more alterations within the gene sequences of the genes involved in regulated secretion. Such alterations include both chromosomal alterations and sequence alterations selected from the group consisting of a translocation, an inversion, a deletion, an insertion or a substitution. In a particular embodiment the screening method will concentrate on identifying alterations which result in a reduction or loss of function of one or more genes encoding proteins involved in regulated secretion. A more particular embodiment relates to the detection of one or more alterations in one or more, most particularly in two or more genes which alterations result in an increase in regulated secretion. A further particular embodiment of the invention relates to the detection of an reduced expression of negative regulators of regulated secretion and/or the detection of an increased expression of positive regulators of gene expression. Particularly, in this regard, detection of alteration in genes involved in the secretion of large core dense vesicles (LCDVs) are envisaged. Most particularly these genes are selected from the group consisting of NBEA, amisyn, SCAMP5 and C10orf74.

Alternatively detection of aberrant expression is envisaged based on detection of altered expression of the gene products of one or more genes encoding proteins involved in regulated secretion using specific ligands, more particularly labelled ligands or antibodies.

Another aspect of the invention provides a method of screening for a therapeutic agents for use in the prevention and/or treatment patients diagnosed as having a neural system disorder, more particularly an autistic spectrum disorder, which method comprises: (A) providing an isolated cell comprising one or more genes involved in regulated secretion (B) introducing to the cell a agent to be screened; and (C) determining whether said agent influences or modulates regulated secretion by the isolated cell;

According to a particular embodiment, in this screening method, the one or more genes involved in regulated secretion are modified or the normal functioning of the gene product of the one or more genes is inhibited. This can be achieved by using antisense, RNAi, homologous recombination or transposons of wild-type genes. Alternatively the cell can comprise one or more genes involved in regulated secretion which are functionally altered versions of wild-type genes. According to a particular embodiment these functionally altered versions are exogenous and optionally heterologous to the cell.

A specific aspect of the invention relates to the screening for a therapeutic compound capable of reducing excessive regulated secretion. Thus, the model developed for such screening particularly comprises genes that have been modified in such a way so as to result in increased regulated secretion. A particular embodiment of the invention relates to screening methods based on detecting aberrant expression of one or more negative regulators of regulated secretion such as, but not limited to neurobeachin, SCAMP5 and SCAMP4, amisyn and other I-SNAREs (inhibitory SNAREs) such as tomosyn and synaptophysin, and C10orf74 and other Rab effectors or effector partners. Yet a further aspect of the present invention provides novel genes for use in the testing or screening of an animal for a neural system disorder, more particularly an autistic spectrum disorder or a predisposition thereto. Thus this aspect provides methods of screening comprising detecting aberrant expression of NBEA and/or amisyn and/or SCAMP5 and/or C10orf74, most particularly screening expression of oner or more of C10orf74, amysin or SCAMP5. Such methods can consist of detecting alterations in at least one, more particularly at least two of these genes, wherein the alteration is a chromosomal alteration or a sequence alteration selected from the group consisting of a translocation, an inversion, a deletion, an insertion or a substitution. Different methods for detecting alterations in the novel genes of the present invention at either the DNA, RNA or protein level are envisaged including detected by hybridisation with a labelled probe. Particular methods include the steps of: (A) extraction of the chromosomal material from said sample, (B) amplification of the chromosomal material using PCR; (C) optionally, sequencing said material; and (D) determining the presence of an alteration in said nucleotide sequence. Alternatively, the detection of aberrant expression is carried out using specific ligands to detect the gene product.

Yet a further aspect of the present invention provides novel polynucleotide sequences comprising the amisyn, SCAMP5, or C10orf74 sequence which comprise an alteration, more particularly alterations resulting in a loss or reduction of function. More specifically, such alterations are envisaged to be selected from the group consisting of a) a substitution, b) a deletion, d) an insertion, or e) one of a chromosomal inversion, a translocation or deletion. The invention further provides vectors and cells comprising the novel nucleotides of the present invention. Additionally the invention provides for the use of a polynucleotide sequence of the wild-type C10orf74 and/or amisyn and/or SCAMP5 gene or a variant C10orf74 and/or amisyn and/or SCAMP5 gene for the diagnosis of a neural system disorder or the predisposition to a neural system disorder, more particularly an autism spectrum disorder in an animal, based on a biological sample of said animal.

The present invention further provides probes and kits for the identification of individuals having a neural system disorder, more particularly an autism spectrum disorder or a predisposition thereto, as well as probes and kits for the identification of patients, having been diagnosed with an autistic spectrum disorder, which are susceptible to the treatment with a compound capable of modulating regulated secretion. Specific embodiment of such kits comprise probes capable of specifically detecting one or more genes selected from the group consisting of neurobeachin, amisyn, SCAMP5 and C10orf74.

Yet another aspect of the present invention provides a method of screening for a therapeutic agents for use in the treatment or therapy of a neural system disorder comprising: (A) providing an engineered yeast cell, comprising an introduced nucleotide sequence comprising C10orf74 and/or amisyn and/or SCAMP5 gene or an allelic variant, minigene, a synthetic gene or a homologue thereof; (B) introducing to the cell a compound, chemical signal or agent to be screened; and (C) correlating the change in said cell with the activity of the compound, chemical signal or agent.

Additionally, the present invention relates to the use of an inhibitor of regulated secretion in the manufacture of a medicament for the treatment of patients which have been diagnosed with autism spectrum disorder and having aberrant expression of genes involved in regulated secretion.

### DETAILED DESCRIPTION

The present invention is based on the identification of several candidate genes correlated with the onset and development of autism. In the Centre of Human Genetics in Leuven, during the past years, 525 individuals with autism were karyotyped. Among them, 5 patients were found who carry a de novo balanced chromosomal aberration. At least four of these chromosomal aberrations were found to affect the expression of genes involved in regulated secretion.

The term ‘regulated secretion’ as used herein refers to the release of secretory products produced and stored within the cell upon stimulation with a relevant chemical or electrical signal. Regulated secretion includes the secretion by both large dense-core vesicles (LDCVs; 70-100 nm in diameter) and small synaptic vesicles (SSVs; ˜40 nm in diameter).

The present invention relates to genes encoding proteins involved in regulated secretion, and their use in the diagnostics and therapeutic diagnostics. According to the present invention proteins involved in regulated secretion include proteins which are involved in the different processes of the vesicle exocytosis machinery, which are sometimes referred to as docking, priming, fusion and endocytosis. According to a particular embodiment, the present invention relates to genes encoding proteins involved in the secretion of LDCVs. A large number of proteins have been identified which are believed to play a role in one or more of these processes, such the soluble N-ethylmaleimide-sensitive factor attachment protein receptors (SNAREs), the RabGTPases, and Munc18/nSec1 proteins. A non-extensive list of proteins involved in vesicle exocytosis includes vesicle associated membrane protein (VAMP2 or synaptobrevin), syntaxin 1, synaptosomal associated protein (SNAP-25), NSF, Sec1, Munc18, Munc 18b, Munc18c, Rab3 (A, B, C, D), Rabphilin, RIM, the Exocyst proteins Sec3p, Sec5p, Sec6p, Sec8p, Sec10p, Sec15p, Exo70p, Exo84p, synaptogagmin, complexin (I and II), Munc 13, tomosyn, amisyn, neurobeachin, . . . .

According to a first aspect of the invention, a subgroup of patients which have been diagnosed with a nervous system disorder can be identified based on aberrant expression of one or more genes involved in regulated secretion. This is of interest in the context of therapeutic diagnosis and pharmacogenomics. According to the present invention, it is possible to predict the response to drugs of patients which have been identified with a neural system disorder, more particularly with an autism spectrum disorder, can based either on an additional phenotypic feature (i.e. by detecting aberrant regulated secretion) or on its genetic background (i.e. by the identification of alterations in genes involved in regulated secretion, either at the DNA or protein level). The present invention makes it possible to identify within the patient group diagnosed with a neural system disorder, those patients which would be susceptible to the treatment with a composition which modulates regulated secretion, more particularly those patients which would be susceptible to the treatment with compositions which reduce excessive regulated secretion. Thus, according to this invention, a subpopulation of patients with NSD is identified, which is of interest in the context of understanding the disease and potential therapy.

Thus, the present invention relates to the detection of aberrant expression of genes encoding proteins involved in regulated secretion. According to a specific embodiment aberrant expression of one or more genes involved in regulated secretion results in an increased secretion. This can be caused by the reduced function of one or more proteins which act as negative regulators in the processes involved in vesicle exocytosis (i.e. the expresion of which negatively influences or reduces regulated secretion). Particular embodiments of proteins which act as negative regulators in the regulated secretion of vesicles according to the present invention include neurobeachin (NBEA), amisyn, SCAMP5 and the protein encoded by the. c10orf74 gene described herein, but also includes tomosyn and synaptophysin, fusogenic SNARE complexes, and SCAMP4 which is (like SCAMP5) a member of the family lacking the N-terminal NPF repeats which are known negative regulators of regulated secretion. As demonstrated herein, aberrant expression of the genes encoding such negative regulators in vitro, such as by inhibition with RNAi, results in an increased regulated secretion of LDCVs.

Alternative embodiments of the present invention relate to detecting aberrant expression of positive regulators of regulated secretion, whereby increased expression of said positive regulators also results in increased regulated secretion.

According to the present invention, aberrant expression of a gene can be the result of an alteration within the gene or can be caused by a change in epigenetic control of said gene, and can be identified either by detecting an alteration in the sequence of the gene itself or by detecting an altered expression of the gene product, either directly or based on its function. Thus, in the context of the present invention aberrant expression of a gene encoding a protein involved in regulated secretion can, according to a particular embodiment, be determined by measuring regulated secretion.

According to a particular embodiment of the present invention, the aberrant expression of genes involved in regulated secretion is determined based on the effect on regulated secretion as observed in cells in which regulated secretion occurs, such as but not limited to neural cells, endocrine cells, platelets . . . . A particular embodiment of the present invention relates to the determination of regulated secretion e.g. in neural cells or platelets. Regulated secretion can be assessed e.g. on isolated cells in vitro using different methods such as quantative determination of the secretion products e.g. by specific immunodetection (such as in ELISA), or incorporation of 3H and detection of secretion of 3H-labelled products, aggregation with agonists (e.g epinephrine, ADP, collagen) or morphological analysis, e.g. of the density of dense granules (using electronmicroscopy). Secretion assays for platelets have been described for instance by Crosby and Poole, (Platelet dense-granule secretion: the [3H]-5-HT secretion assay, Methods Mol. Biol. 2004, 272:95-96; Chen et al. 2000, Blood 95:921-929; Derreck et al. 1997, Biochem J. 325:309-313). Similary, the secretion of primary neurons can be assessed, optionally making use of stimulatory agents.

According to an alternative embodiment of the present invention, aberrant expression of genes encoding proteins involved in regulated secretion is determined by identification of alterations in the sequence of such genes or other genes which mediate their expression.

When referring to a particular gene such as, but not limited to, the neurobeachin, amisyn, SCAMP5 or the C10orf74 gene or locus herein it is intended to include coding sequences, intervening sequences and regulatory sequences, including the promoter, controlling transcription and/or translation. Reference to neurobeachin, amisyn, SCAMP5 or C10orf74 locus is intended to include all allelic variations of the DNA sequence. The wild-type gene as referred to herein corresponds to the gene sequence as occurring in the general population not affected by a neural system disorder. In this context, when referring to an entry number of a Genbank or similar database, it will be understood by the skilled person that, for sequence analysis purposes, the most recent update of the Genbank entry will be the most useful. Small variations between the sequence of the Genbank entry as publicly available on the filing date, the publication date or the grant date of the present application are believed to be self-correcting errors, which can be identified by the skilled person. Polymorphisms occurring in the wild-type gene can be distinguished from alterations in the gene which are indicative of a neural system disorder or a predisposition to a neural system disorder by segregation/mutation analysis.

As used herein, the term “minigene” refers to a heterologous gene construct wherein one or more nonessential segments of a gene are deleted with respect to the naturally-occurring gene. Typically, deleted segments are intronic sequences of at least about 100 basepairs to several kilobases, and may span up to several tens of kilobases or more. Isolation and manipulation of large (i.e., greater than about 50 kilobases) targeting constructs is frequently difficult and may reduce the efficiency of transferring the targeting construct into a host cell. Thus, it is frequently desirable to reduce the size of a targeting construct by deleting one or more nonessential portions of the gene. Typically, intronic sequences that do not encompass essential regulatory elements may be deleted. Frequently, if convenient restriction sites bound a nonessential intronic sequence of a cloned gene sequence, a deletion of the intronic sequence may be produced by: (1) digesting the cloned DNA with the appropriate restriction enzymes, (2) separating the restriction fragments (e.g., by electrophoresis), (3) isolating the restriction fragments encompassing the essential exons and regulatory elements, and (4) ligating the isolated restriction fragments to form a minigene wherein the exons are in the same linear order as is present in the germline copy of the naturally-occurring gene. Alternate methods for producing a minigene will be apparent to those of skill in the art (e.g., ligation of partial genomic clones, which encompass essential exons but which lack portions of intronic sequence). Most typically, the gene segments comprising a minigene will be arranged in the same linear order as is present in the germline gene, however, this will not always be the case. Some desired regulatory elements (e.g., enhancers, silencers) may be relatively position-insensitive, so that the regulatory element will function correctly even if positioned differently in a minigene than in the corresponding germline gene. For example, an enhancer may be located at a different distance from a promoter, in a different orientation, and/or in a different linear order. For example, an enhancer that is located 3′ to a promoter in germline configuration might be located 5′ to the promoter in a minigene. Similarly, some genes may have exons, which are alternatively spliced, at the RNA level, and thus a minigene may have fewer exons and/or exons in a different linear order than the corresponding germline gene and still encode a functional gene product. A cDNA encoding a gene product may also be used to construct a minigene. However, since it is often desirable that the heterologous minigene be expressed similarly to the cognate naturally-occurring non-human gene, transcription of a cDNA minigene typically is driven by a linked gene promoter and enhancer from the naturally-occurring gene. Frequently, such minigene may comprise a transcriptional regulatory sequence (e.g., promoter and/or enhancer) that confers neuron-specific or CNS-specific transcription of the minigene.

The term “Regulatory sequences” refers to a sequence which affects the expression of the gene (including transcription of the gene, and translation, splicing, stability or the like of the messenger RNA). Regulatory sequences are most often, but not necessarily, comprised within a region of 100 kb 5′ or 3′ of the coding region of a gene.

According to this aspect of the present invention tools are provided for the detection of a neural system disorder or a predisposition to a neural system disorder in an animal, more particularly a human based on the detection of an alteration in of one or more genes involved in regulated secretion. According to a particular embodiment such genes encode proteins which are negative regulators of regulated secretion, such as tomosyn, neurobeachin, amisyn, SCAMP5 gene and/or of the C10Orf74 gene. The association of neurobeachin with autism is described in WO2004/033717. For the latter three genes a link between a functional alteration and the occurrence of a neural system disorder is described herein established.

The term “alteration” when referring to a wild-type gene relates to any kind of mutation, such as, but not limited to deletion, insertion and point mutation (or substitution) in the coding and non-coding regions of the wild-type gene. A deletion can encompass the entire wild-type gene or a portion thereof. Point mutations may result in stop codons, frameshift mutations or amino acid substitutions. Germline mutations are mutations which are inherited and can be detected in any tissue of the body. A “functional alteration” as used herein relates to an alteration in the gene which affects transcription and/or translation of a gene encoding a protein involved in regulated secretion, thus modifying the expression level of the gene product, or leading to a gene product which is non-functional or only partly functional. Functional alterations in the coding regions can be frame-shift mutations or mutations resulting in the occurrence of a stop codon. Functional alterations in the non-coding regions include, but are not limited to point mutations such as in the promoter of the gene or in the splice-junctions, leading to loss or diminution of expression of the mRNA, loss of proper RNA processing, decreased mRNA stability or translation efficiency. It will be understood by the skilled person that an alteration in a gene also includes changes at the chromosomal level (‘chromosomal alterations’) which affect the gene of interest such as translocations, deletions, insertions and inversions. These chromosomal alterations are usually reflected in the nucleotide sequence. Thus an ‘altered or variant gene’ as used herein refers to a gene encoding a protein involved in regulated secretion will be understood to refer to a gene which comprises an alteration as described above. Similarly, a polynucleotide ‘corresponding to’ the sequence of the wildtype gene’ is used herein to refer to a polynucleotide sequence having a sequence identical to the wildtype sequence, taking into account possible polymorphisms observed in the wild-type gene.

Detecting the presence of an alteration of a gene can be achieved in different ways known to the skilled person and can be based on the detection of aberrant expression levels or the specific detection of a change in the nucleotide sequence. It includes detection at the DNA or RNA level using techniques described in the art, such as but not limited to, fluorescent in situ hybridization (FISH), direct DNA sequencing, PCR amplification, pulsed field gel electrophoresis (PFGE) analysis, denaturing gradient gel electrophoresis (DGGE) (Wartell et al., 1990; Sheffield et al., 1989), Northern or Southern blot analysis, single stranded conformation analysis (SSCA) (Orita et al., 1989), RNase protection assay (Finkelstein et. al., 1990; Kinszler et al., 1991), allele-specific oligonucleotide (ASO) (Conner et al., 1983), the use of proteins which recognize nucleotide mismatches, such as the E. coli mutS protein (Modrich, 1991), dot blot analysis, allele-specific PCR (Rano & Kidd, 1989) and PCR-SSCP.

A rapid preliminary analysis to detect polymorphisms in DNA sequences can be performed by looking at a series of Southern blots of DNA of normal individuals of patients with the neural system disease cut with one or more restriction enzymes. The presence of fragments which hybridize with a gene-specific probe which are different in length from the control DNA, is indicative of a possible mutation.

Direct DNA sequencing, either manual sequencing or automated fluorescent sequencing can detect sequence variation. Manual sequencing is very labor-intensive, mutations in the coding sequence of a gene are usually detected. Another approach is the single-stranded conformation polymorphism assay (SSCA) (Orita et al., 1989). This method does not detect all sequence changes, especially if the DNA fragment size is greater than 200 bp, but can be optimized to detect most DNA sequence variation. The fragments which have shifted mobility on SSCA gels are sequenced to determine the exact nature of the DNA sequence variation. Other approaches include clamped denaturing gel electrophoresis (CDGE) (Sheffield et al., 1991), heteroduplex analysis (HA) (White et al., 1992) and chemical mismatch cleavage (CMC) (Grompe et al., 1989). For the detection of large deletions, duplications, insertions, or a regulatory mutation which affects transcription or translation of the protein, protein truncation assays can also be used.

Detection of point mutations may be accomplished by molecular cloning of the allele(s) of the gene of interest and sequencing the allele(s) using techniques well known in the art. Alternatively, the gene sequences can be amplified directly from a genomic DNA preparation from the tumor tissue, using known techniques. The DNA sequence of the amplified sequences can then be determined.

Thus according to this aspect of the invention, aberrant gene expression is identified by detecting an alteration at the DNA and/or RNA level of one or more genes, particularly at least two genes involved in regulated secretion (such as, but not limited to the genes described above). According to a specific embodiment aberrant regulated secretion is determined by analysis of the expression of genes which upon functional alteration result in increased secretion (i.e. genes encoding negative regulators of the regulated secretion pathway). Most particularly, according to the present invention aberrant regulated secretion is determined by analysis of the expression of one or more genes selected from the group consisting of amisyn, SCAMP5, neurobeachin and the C10orf74 gene described herein. Most particularly, aberrant secretion can be assayed by identifying functional alterations in one or more of these genes.

For it to be relevant, an alteration in the genes, more particularly in the neurobeachin, amisyn, SCAMP5 and/or the C10orf74 gene expression should in some way disrupt gene product function or expression. In addition it can be considered whether the alteration is present in individuals from the kindred who carry the altered neurobeachin, amisyn, SCAMP5 and/or C10orf74 haplotype and absent in other members of the kindred, and is rare in the general population.

Thus, in accordance with the above-described embodiments the present invention provides probes, primers and ligands for use in the detection of aberrant expression of the neurobeachin, amisyn, SCAMP5 and/or C10orf74 gene.

The term “probe” as used herein refers to a polynucleotide which under stringent to moderately stringent hybridization conditions, forms a stable hybrid with a target sequence, based on the complementarity to the target sequence. According to a particular embodiment of the present invention the target sequence is the wild-type or altered genes, more particularly the neurobeachin, amisyn, SCAMP5 or C10orf74 gene. According to a more particular embodiment, the probe of the present invention will allow specific detection of one or more alterations within the neurobeachin, amisyn, SCAMP5 or C10orf74 genes. Alternatively, the probe will form a stable hybrid with a target sequence common to both the wild-type and one or more altered genes. The length of the probe may vary (with a minimum of 5 nucleotides, preferably minimally 12 nucleotides) depending on the degree of complementarity with the target sequence. A labeled probe as used herein refers to a probe to which a label or reporter molecule has been attached, allowing detection and/or isolation of the hybridized probe and its target sequence. Methods for labeling probes are known to the skilled person and have been described e.g. in Sambrook et al., 1989 and Ausubel et al., 1992.

In order to allow formation of a stable hybrid with the target sequence under standard hybridising conditions, the probes provided in the context of the present invention are at least 70%, more particularly 80%, even more particularly 90%, most particularly more than 90% complementary to the target sequence. Deviations from complementarity may be introduced in particular embodiments of the present invention, e.g. in order to include variability based on the redundancy of the genetic code, to introduce particular restriction sites, or to optimize expression.

The probes of the present invention can be recombinant probes, can be chemically synthesized or can be derived from naturally occurring polynucleotides, according to methods known to the person skilled in the art.

According to the present invention, one or more of such probes can be packaged in the form of a kit, for use in the detection of aberrant expression of one or more genes encoding proteins involved in regulated synthesis. Depending on the nature of the detection method to be performed, the exact nature of the probe may vary, but this is within the ambit of the skilled person. According a particular embodiment the invention relates to a kit for use in the diagnosis of an autism spectrum disorder or a predisposition thereto, comprising probes for the detection of at least two genes involved in regulated secretion. According to another embodiment of the present invention kits are provided comprising probes for the detection of one or more genes involved in regulated secretion, whereby at least one of said genes is amisyn, SCAMP5 or c10orf74. A further particular embodiment relates to a kit according to the invention wherein said one or more genes are selected from the group consisting of amisyn, SCAMP5, neurobeachin and c10orf74.

According to a particular embodiment of the invention, the method for detecting a neural system disorder in an animal comprises the provision and optionally preparation of a biological sample and detecting the altered expression of one or more genes, preferably at least two genes involved in regulated secretion. More particularly the present invention provides methods for detecting altered neurobeachin, and/or amisyn, and/or SCAMP5 and/or C10orf74 gene expression therein. According to a particular embodiment the biological sample is a nucleic acid sample and detection is based on identification of an alteration in the expression of one or more genes which negatively regulate regulated secretion such as, but not limited to neurobeachin, tomysin, amisyn, SCAMP5 and/or the C10orf74 gene. Detection of an alteration is performed by comparison with the respective wildtype sequences. Most methods of detection based on nucleotide acid sequences will involve the amplification of the relevant gene locus, such as by polymerase chain reaction (PCR) or non-PCR based methods. The amplified nucleic acid can then be analyzed for size, sequenced or identified based on detection with a labeled probe or with a probe which can itself be detected e.g. by binding with a labeled ligand or probe. Suitable labels are known in the skilled person and include, but are not limited to fluorescent labels, radioactive labels, luminescent labels, enzymatic labels, biotin, antibodies, metal-based labels etc..

Specific embodiments of the methods of the present invention comprise the steps of detecting the presence of an alteration in the sequence of a gene in a DNA sample, most particularly at least two genes encoding proteins involved in regulated secretion, wherein presence of an alteration is indicative of a neural system disorder or a predisposition to a neural system disorder. Thus, presence of the alteration is correlated with neural system disorders or a potential for neural system disorders, more particularly autism.

Specific embodiments of the methods of the present invention comprise the step of performing linkage analysis, to determine the correlation of the aberrant expression of the gene involved in regulated secretion, with the disease. Thus, according to a particular embodiment the method of detection comprises the step of correlating the alteration in the genes involved in regulated secretion with a neural system disorder or a potential for a neural system disorder.

Particular embodiments of the present invention relate to detection methods which employ more than one nucleic acid probe capable of detecting the genes involved in regulated secretion according to the present invention, more particularly the neurobeachin, amisyn, SCAMP5 and/or C10orf74 genes, respectively or variants thereof. This can be of interest to ensure the detection of the allelic variations of the gene or the simultaneous detection of different mutations. According to a particular embodiment of the present invention, the probes for use in the detection correspond to the sequences described herein.

Specific embodiments of the methods of the present invention comprise the steps of (providing the sample and) detecting altered expression of genes involved in C10orf74 and/or SCAMP5 gene product in a sample of a patient, wherein a qualitative or quantitative change in the expression of the gene product is indicative of a neural system disorder or a predisposition to a neural system disorder in the patient from which the sample originates. Thus, altered expression of the gene product is correlated with neural system disorders or a potential for neural system disorders, more particularly autism.

According to a particular embodiment of the present invention, detection of a neural system disorder is achieved by using a probe or polynucleotide sequence which is capable of specifically hybridizing to an altered or variant of the gene encoding a protein involved in regulated secretion. More specifically, the altered gene is characterized by a deletion, insertion or base substitution.

Alternatively, detecting the presence of an alteration in a gene encoding a protein involved in regulated secretion can be performed by detection of an alteration in the expression of the wild-type gene product. Alteration of wild-type gene product expression can be either altered (i.e. aberrant) expression levels of the wild-type gene product or (optionally also altered) expression of an aberrant gene product, i.e. a gene product not corresponding to the wild-type gene product. More particularly, such aberrant gene product does not or to a lesser extent perform the function of the wild-type gene product. Detection of these phenotypes can be achieved using specific ligands, such as monoclonal or polyclonal antibodies directed to the wild-type gene product of one or more genes involved in regulated secretion (such as, but not limited to the genes described above). Alternatively, specific ligands such as antibodies directed to mutant gene products can be used to detect specific alterations in a gene.

According to a particular embodiment such a specific ligand is a monoclonal or polyclonal antibody, or a fragment thereof (such as, but not limited to Fab, F(ab)2, scFvs), or a homologue thereof (e.g. the single chain antibodies obtained from certain animals such as camels and lamas). Methods for obtaining such antibodies or antibody fragments are known to the skilled person and can be based on synthetically prepared peptides or naturally isolated proteins or peptides. Methods for the immunological detection of an antigen are well known in the art and include but are not limited to ELISAs, RIAs, or Western blots of cell lysates or immunohistochemical assays on intact cells.

Thus according to this aspect of the invention, aberrant gene expression is identified by detecting an alteration at the protein level of genes involved in regulated secretion (such as, but not limited to the genes described above). A particular embodiment relates to the determination of reduced expression of one or more, particularly at least two genes encoding proteins which act as negative regulators of regulated secretion or increased expression of genes encoding proteins which act as positive regulator of regulated secretion. According to a specific embodiment aberrant regulated secretion is determined by analysis of the expression of genes which upon functional alteration (such as truncation), cause increased secretion (i.e. negative regulators of the regulated secretion pathway). Most particularly, according to the present invention aberrant gene expression is determined by analysis of the expression of one or more gene products selected from the group consisting of amisyn, neurobeachin, SCAMP5 and the C10orf74 gene product as described herein. Reduced expression of one or more of these proteins will result in an upregulation of regulated secretion which according to the present invention is associated with autism, more particularly a subgroup of the patients which are autistic or are believed to have a predisposition to autism and which are susceptible to the treatment with pharmaceutical compositions which modulate such increase in regulated secretion. Examples of specific antibodies to amisyn and neurobeachin and their use in the detection of expression of these gene products in cell lines are described herein.

Thus, a particular embodiment of the first aspect of the present invention relates to identifying individuals having aberrant expression of one or more genes involved in regulated secretion or a predisposition thereto. Most particularly, the present invention relates to identifying, upon a clinical diagnosis of a neural system disorder, more particularly an autism spectrum disorder, those patients which are characterized by aberrant regulated secretion or which display a predisposition thereto, more particularly those patients which are characterized by an increased regulated secretion, and which would thus benefit from medication which modulates, more particularly suppresses such aberrant regulated secretion.

In a particular embodiment such identification is based on the testing of a biological sample of said individual or patients. The term ‘biological sample’ as used herein refers to a sample from an animal or human body, i.e. including a sample from any body tissue as well as samples obtained from serum, stool, urine and sputum. For the analysis of DNA, the biological sample will preferably contain differentiated or undifferentiated cells, from which DNA can be extracted. An exemplary way of obtaining a DNA sample is by drawing blood from the body of the animal or person to be tested, separating out the cells of the blood and extracting the DNA therefrom. The preparation of a biological sample for DNA analysis is known to the skilled person. For specific applications, preparation of the DNA for further analysis may require treatments such as denaturation, restriction digestion, electrophoresis etc.. Alternatively, a biological sample will be used to determine the secretion of intact cells (such as platelets) or to identify cellular proteins upon fractionation.

The methods for detecting aberrant expression of one or more genes encoding proteins involved in regulated secretion according to the present invention can be performed on biological samples of different stages of development. Prenatal diagnosis can be performed on fetal cells, placental cells or amniotic cells.

The present invention relates to the identification and treatment of patients which have or are susceptible to an Neural system disorder (NSD). An NSD is characterized by the disruption of a process in the nervous system. According to a particular embodiment, the NSD referred to herein is a pervasive development disorder (PDD) or an autism/autistic spectrum disorder (ASD), which includes autism, Asperger syndrome and conditions referred to as PDD Not Otherwise Specified. A particular embodiment of the present invention relates to the detection, therapy prevention of autism or detection, therapy and prevention of a subgroup of patients which have been clinically diagnosed as having and ASD.

According to a particular embodiment the neural system disorder considered in the present invention is a neural system disorder which results from a brain anomaly, more particularly an abnormality in the cerebellum, most particularly characterized by a decreased number of Purkinje cells.

According to a further particular embodiment of the invention the neural system disorder is characterised in that neural system disorder results from a disturbed the glutamate neurotransmitter system. Additionally or alternatively is characterized by reduced levels of the anti-apoptotic protein bcl2 in the brain. Additionally or alternatively, the neural system disorder is associated with any or several symptoms selected from the group consisting of disturbed cognitive functions, disturbed emotional control, disturbed in motor control.

According to one aspect of the invention, within the patients diagnosed as having a neural system disorder, a patient group is identified which is susceptible to treatment with a modulator of regulated secretion. Susceptibility to a drug or pharmaceutical composition means that the likelihood of a positive response of the patients' body to that drug is increased, particularly to above 50%, most particularly to above 60%. A response is considered positive when there is an improvement in one or more of the symptoms indicative of the neural system disorder such as but not limited to disturbed cognitive functions, disturbed emotional control, disturbed in motor control.

More specifically, symptoms of an autism spectrum disorder include the following
qualitative impairments in communication as manifested by: a) delay in, or total lack of, the development of spoken language (not accompanied by an attempt to compensate through alternative modes of communication such as gesture or mime) b) in individuals with adequate speech, marked impairment in the ability to initiate or sustain a conversation with others; c) stereotyped and repetitive use of language or idiosyncratic language d) lack of varied, spontaneous make-believe play or social imitative play appropriate to developmental level
qualitative impairment in social interaction, as manifested by a) marked impairments in the use of multiple nonverbal behaviors such as eye-to-eye gaze, facial expression, body posture, and gestures to regulate social interaction, b) failure to develop peer relationships appropriate to developmental level, c) a lack of spontaneous seeking to share enjoyment, interests, or achievements with other people, (e.g., by a lack of showing, bringing, or pointing out objects of interest to other people), d) lack of social or emotional reciprocity (note: in the description, it gives the following as examples: not actively participating in simple social play or games, preferring solitary activities, or involving others in activities only as tools or “mechanical” aids)
restricted repetitive and stereotyped patterns of behavior, interests and activities, as manifested by: a) encompassing preoccupation with one or more stereotyped and restricted patterns of interest that is abnormal either in intensity or focus; b) apparently inflexible adherence to specific, nonfunctional routines or rituals; c) stereotyped and repetitive motor mannerisms (e.g hand or finger flapping or twisting, or complex whole-body movements); d) persistent preoccupation with parts of objects

Moreover, in children younger than 3 years of age, typical symptoms of autism spectrum disorder include the occurrence of delays or abnormal functioning in the areas of social interaction, language as used in social communication and/or symbolic or imaginative play

Summarized, the symptoms of an autism spectrum disorder, most particularly in young children, are language delay (language regression), eye contact or socialization impairment, lack of pleasure with regard to being touched, and indifference to surroundings.

Screening Tools for Pharmaceutical Compositions Useful in the Treatment of NSDs

According to yet another aspect of the present invention, tools are provided for the identification of pharmaceutical compositions useful in the treatment of patients diagnosed with a neural system disorder, more particularly with an autism spectrum disorder.

Compounds capable of modifying regulated secretion can be identified using assays for regulated secretion, such as the assay making use of cell-lines capable of regulated secretion, more particularly neural or endocrine cell-lines. Permeabilized PC12 phaeochromocytoma cells and bovine chromaffin cells have been shown to provide models for the mechanism by which dense core granules containing polypeptides and hormones are generated in neuroendocrine cells (Martin, T. F. J. (1997) Trends Cell Biol. 7, 271-276; Burgoyne, R. D., and Morgan, A. (1998) BioEssays 20, 328-335).

According to a particular embodiment, the assay is performed using a the beta-TC3 cell-line or using neuronal cells. Secretion can be monitored in different ways, e.g. by detection of labeled secretion products, by detection of secretion products using specific ligands, by morphological examination of the cells, more particularly of the secretory granules, etc. According to a further particular embodiment, the expression of one or more genes involved in regulatory secretion is inhibited in the cells, e.g. by using cells of knock-out mice or by silencing one or more genes by methods known in the art.

Additionally or alternatively, compounds capable of reducing or inhibiting excessive regulated secretion by direct or indirect action on genes involved in regulated secretion can be screened by making use of a yeast model. Many of the genes involved in regulated secretion have yeast counterparts (Finger and Novick J. Cell Biol. 1998, 142:609-612). Thus, by using one or more genes involved in regulated secretion which comprises a functional alteration, to replace the corresponding yeast gene, compounds can be screened which are capable of reducing the effect of the presence of the altered gene on the yeast cell.

According to the present invention such model systems are based on cells wherein the expression of one or more genes involved in regulated secretion is modified or the normal functioning of the gene product of the one ore more genes is inhibited. Inhibition of gene expression can be obtained in different ways known to the skilled person such as, but not limited to homologous recombination, transposons, antisense RNAi. The genes involved in regulated secretion can be endogenous to the organism from which the cell originates (e.g. human, bovine, yeast), or can be exogenous and introduced by recombinant methods. According to the present invention, the one or more genes can be wild-type genes. According to a particular embodiment of the present invention, at least one of the genes encoding a protein involved in regulated secretion is a functionally altered version of the wild-type gene. According to a particular embodiment at least one of the one or more genes involved in regulated secretion which forms the basis of the model of regulated expression is selected from the group consisting of tomosyn, amisyn, SCAMP5, C10ORF74 and neurobeachin.

A particular embodiment of the present invention relates to the use of models wherein regulated secretion is increased, i.e. above normal levels and can be considered excessive. An increase in regulated secretion refers to an increase of one or more secretion products of the regulated secretion pathways by at least 10%, more particularly by at least 20%, most particularly by at least 50%.

Additionally or alternatively, compounds capable of reducing or inhibiting regulated secretion by direct or indirect action on genes involved in regulated secretion can be screened by making use of other model organisms in which genes involved in regulated secretion similar to those found in humans have been identified, such as but not limited to Drosophila, C. elegans and Danio rero.

Methods of Treatment and/or Prevention of NSD Patients

The present invention is based on the observation that the occurrence of autism spectrum disorder is, in at least a subgroup of patients, linked to an increase in regulated secretion. Thus, according to yet another embodiment of the present invention, pharmaceutical compositions are provided for the treatment of patients diagnosed with a neural system disorder, more particularly an autism spectrum disorder, which are characterized by such an aberrant regulated secretion. According to a particular embodiment of this aspect of the invention pharmaceutical compositions are provided for the treatment and prevention of patients diagnosed with or susceptible to an autistic spectrum disorder which are characterized with an aberrant regulated secretion or a predisposition thereto. The identification of patients which have or are predisposed to an aberrant regulated secretion can be done using one of the methods described above.

Compositions which modulate regulated secretion include compounds which are capable of reducing secretion of LCDVs and/or SSVs.

Compounds which have been identified in the art to modulate, more particularly reduce or inhibit secretion include, but are not limited to C2 domain peptides of synaptotagmin (Bommert et al., 1993, Nature 363:163-165), Rnai of synaptotagmin IX, KCL, 4-aminopyridine, botulinum toxin c1.

Alternatively, it can be envisaged that an increased regulated secretion can be treated by targeting genes or gene products which act as positive regulators of regulated secretion. Compounds which have been identified in the art to act as positive regulators of regulated secretion include, but are not limited to synaptotagmin (Syt)I, Syt IV and Syt IX.

Additionally, compositions which modulate regulated secretion include those compounds identified by the screening methods described above.

Identification of Genes, the Disruption of Which is Linked to NSD

From previous studies, the neurobeachin gene has been identified as an autism susceptibility gene. The link between vesicle trafficking and neural system disorders had however not been made.

The mouse neurobeachin cDNA (mNbea) and the corresponding protein (mNBEA) are known (Wang, X., et al. J. Neurosci. 20 (23), 8551-8565 (2000)) DBSOURCE Accession Number Y18276.1; see also “The neurobeachin gene (Nbea) identifies a new region of homology between mouse central chromosome 3 and human chromosome 13q13” (Gilbert, D. J. Mamm. Genome 10 (10), 1030-1031 (1999)). The full-length cDNA sequence of the human orthologue of neurobeachin is known DBSOURCE Accession Number AF467288 or NM_{—}015678.

The neurobeachin gene maps at 26.87 cM near marker D13S624, at the boundary of chromosome 13q13.2-13.3. It is of particular interest that this falls within the 19 cM region that was identified as a candidate region for autism on chromosome 13q (Barrett S. Am J Med Genet 1999, 88:609-15). A maximum MMLS/het score of 2.3 was found in this region between markers D13S217 (17.21 cM) and D13S1229 (21.51 cM). Approximately one-third of the families were found to link to this locus under a recessive model for autism. A follow up study, incorporating information on proband and parental language development, reinforced the finding that this locus may harbour an autism susceptibility gene. The precise genomic position of common fragile site FRA13A was recently positioned to a limited 650 kb region within the neurobeachin (NBEA) gene (Savelyeva et al., 2005, Hum Genet 22:1-8).

According to yet another aspect of the invention, three novel genes are identified which encode proteins involved in regulated secretion, the aberrant expression of which is linked to the occurrence of or a predisposition to a NSD, more particularly an ASD. These genes are the amisyn gene, the SCAMP5 gene and c10orf74.

Amisyn or syntaxin binding protein 6 (STXBP6) has been identified to bind components of the SNARE complex and is known to be involved in regulating SNARE complex formation (Scales et al., 2002 J. Biol. Chem. 277: 28271-28279). The amisyn gene (NCBI mRNA BC067278 [NM_{—}014178]) consists of 6 exons spanning 238 kb of the human genome at 14q12. There is evidence for an alternative transcript (NCBI mRNA BC009499) in which exon 1 is replaced by two alternative exons. Both transcripts use the ATG start in their respective second and third exon, and contain a coding sequence (CDS) of 630 bp (210 M). Based on alignments of AF391153 (CDS of 714 bp, 238 AA) against the human genome (BLAT search; Kent W J et al. Genome Res 2002;12(6):996-1006), it is hypothesized that sequencing errors at the 5′ end of this mRNA suggested a non-existing ATG start codon 36 bp upstream of the ATG start of BC067278 and BC009499. Interestingly, a 5′ truncated partial mRNA sequence (NCBI AL834346) corresponding with the HCDI gene, as amisyn was called at the time (Dec. 7, 2002), suggests the presence of a genomic 17 bp polyA stretch. As a consequence, the gene gives rise to transcripts of approximately 4.0 kb, which is about 2450 bp longer than suggested by BC067278 and BC009499.

According to a particular embodiment of the present invention the wild-type amisyn gene corresponds to the full length gene the sequence of which is as deposited in Genbank accession number AF391153, BC067278 and BC009499. Interestingly, a 5′ truncated partial mRNA sequence (NCBI AL834346) corresponding with the HCDI gene, as amisyn was called at the time (Dec. 7, 2002), suggests the presence of a genomic 17 bp polyA stretch. As a consequence, the gene gives rise to transcripts of approximately 4.0 kb, which is about 2450 bp longer than suggested by BC067278 and BC009499. Amisyn genes have been identified in other species including Xenopus [BC075314], mus musculus [BC009499].

The “amisyn gene product” as used herein is used to describe the protein encoded by the amisyn gene. The human amisyn cDNA codes for a 222 amino acid protein [AAM46624.1] which has been described to contain a VAMP (synaptobrevin)-like coiled coil forming domain which forms SNARE complexes with syntaxin 1 and SNAP25.

The C10orf74 gene has been identified based on a chromosomal inversion observed in an autistic patient, as described herein. According to a particular embodiment of the present invention the wild-type C10Orf74 gene corresponds to the full length gene, the sequence of which is as deposited in Genbank accession number [NM_{—}001001330].

The term “C10orf74 gene product” as used herein is used to describe the protein encoded by the C10orf74 gene. The human C10orf74 cDNA codes for a 255 amino acid protein. The predicted C10orf74 protein has high homology to proteins in several other species, most of which have not been characterized yet. They share a common domain called “TB2/DPI and HVA22” (Pfam PF03134, see FIG. 2). Functional data are available on two homologues, the yeast Yop1p and a plant homologue HVA22 (as detailed in the example section herein).

The SCAMP5 gene has been identified based on a chromosomal translocation, involving chromosomes 1p and 15q, observed in an autistic patient, as described herein. According to a particular embodiment of the present invention the wild-type SCAMP5 gene corresponds to the full length gene, the sequence of which is as deposited in Genbank accession number [NM_{—}138967]. The SCAMP5 gene (NCBI mRNA AL834226) consists of 7 exons spanning 26 kb of the human genome at 15q24.2. SCAMP5 is the brain-specific member of the secretory carrier membrane protein family involved in vesicle budding from trans-Golgi. In general, SCAMPs are composed of a cytoplasmic N-terminal sequence with NPF repeats, four central transmembrane regions (TMRs) and a cytoplasmic tail. In many proteins NPF repeats are binding sites for EH (Eps15 homology) domain proteins, which are involved in clathrin-mediated vesicle budding from the plasma membrane or trans-Golgi complex. In addition, SCAMPs have a conserved basic aromatic segment between TMR 2 and 3, facing the cytosol, called the E-peptide.

The term “SCAMP5 gene product” as used herein is used to describe the protein encoded by the SCAMP5 gene. The gene encodes for a transcript of 3.4 kb, with an ATG start in exon 2, yielding a coding sequence of 708 bp.

Based on the finding that the expression of a number of genes involved in regulated secretion is aberrant in patients diagnosed with a NSD, the linkage of other genes which influence regulated secretion with the etiology and/or pathology of NSD is claimed as a general concept, based on which screening tools and potential therapies can be developed. Other genes which encode proteins which influence regulated secretion, more particularly which act as negative regulators of LDCV secretion include but are not limited to I-SNAREs (inhibitory SNARES) such as tomosyn (NM 139244) and synaptophysin (NM 003179), and other Rab effectors or effector partners.

According to the present invention, tools are provided for the detection, prevention and/or therapy of a neural system disorder or a predisposition to a neural system disorder in an animal, more particularly in a human. More particularly, the neural system disorder considered in the present invention is a neural system disorder which results from a brain anomaly, more particularly an abnormality in the cerebellum, most particularly characterized by a decreased number of Purkinje cells.

According to a particular embodiment of the invention the neural system disorder is characterised in that neural system disorder results from a disturbed the glutamate neurotransmitter system. Additionally or alternatively is characterized by reduced levels of the anti-apoptotic protein bcl2 in the brain. Additionally or alternatively, the neural system disorder is associated with any or several symptoms selected from the group consisting of disturbed cognitive functions, disturbed emotional control, disturbed in motor control.

According to a particular embodiment of the present invention, the neural system disorder referred to herein is a pervasive development disorder (PDD) or an autism spectrum disorder, which includes autism, Asperger syndrome and conditions referred to as PDD Not Otherwise Specified. A particular embodiment of the present invention relates to the detection, therapy and prevention of autism.

According to a particular aspect of the invention tools are provided for the development of compounds of use in the prevention and/or therapy of a neural system disorder.

A particular embodiment of this aspect of the invention relates to the use of a polynucleotide comprising the C10orf74 gene and/or SCAMP5 gene (including any allelic variant thereof) or a fragment thereof encoding C10Orf74 and/or SCAMP5 or a fragment thereof in the manufacture of a medicament for preventing or treating a neural system disorder. The present invention is based on the observation that absence of expression of the C10orf74 gene and/or SCAMP5 products is linked to a neural system disorder. Polynucleotides encoding the C10Orf74 and/or SCAMP5 gene products or active fragments thereof can thus be used directly, i.e. to restore the expression of the gene product, e.g. by gene therapy, or indirectly, e.g. in the production of the C10orf74 and/or SCAMP5 gene product or fragments thereof to restore the level of the C10orf74 and/or SCAMP5 gene product, e.g. by administration.

According to a further aspect of the present invention polynucleotides comprising the C10Orf74 gene and/or SCAMP5 gene or fragments thereof are used in the screening for compounds of use in the prevention and/or treatment of a neural system disorder.

According to an alternative embodiment the C10Orf74 and/or SCAMP5 gene product(s) or a fragment thereof is used to identify potential therapeutic targets involved in neural system disorders. More particularly fragments of the C10Orf74 and/or SCAMP5 gene product corresponding to binding regions of the C10Orf74 protein and/or SCAMP5, respectively are of interest in this regard. According to this embodiment the C10orf74 polypeptide or a fragment thereof and/or the SCAMP5 polypeptide or a fragment thereof are used either unbound (in solution) or bound to a solid support, and are optionally labeled. Alternatively, the polypeptide(s) can be expressed by transformed eukaryotic or prokaryotic host cells. The C10orf74 and/or SCAMP5 polypeptide or fragment thereof can thus be used in binding assays, e.g. the binding of the C10orf74 and/or SCAMP5 polypeptide to another protein. Such an assay can be used to identify targets that interfere with or can modulate the interference with the binding of each of the C10orf74 and/or SCAMP5 polypeptide(s) and other proteins, respectively.

According to another embodiment of this aspect of the invention screening for potential therapeutic agents for the treatment or prevention of neural system disorders is performed based on the activity of the C10orf74 and/or SCAMP5 gene(s) in a prokaryotic or eukaryotic host cell, or in a transgenic (non-human) organism. The C10orf74 and/or SCAMP5 genes can either be the wild-type C10orf74 and/or SCAMP5 genes or an aberrant version thereof. The functional assay of the C10orf74 and/or SCAMP5 genes can be based on their involvement in exocytosis, more particularly in the secretion of large dense-core vesicles (LCDVs), for instance as described in the examples herein or based on the actual secretion of the neurothrophins, such as, but not limited to NGF or BDNF. Alternatively, expression of an aberrant C10orf74 and/or SCAMP5 gene, which results in non-expression of the C10orf74 and/or SCAMP5 gene product, respectively or expression of a non-functional C10orf74 and/or SCAMP5 gene product, can be used to identify agents capable of regulating growth of C10orf74 and/or SCAMP5 defective cells. Alternatively expression of an aberrant C10orf74 and/or SCAMP5 gene in a non-human animal, which results in non-expression of the C10orf74 and/or SCAMP5 gene product(s) or expression of a non-functional C10orf74 and/or SCAMP5 gene product, respectively, can be used to identify agents capable of moderating the symptoms of the neural system disorder, more particularly of autism.

Cells and organisms for use in this aspect of the present invention can be cells comprising an exogenous C10orf74 gene and/or exogenous SCAMP5 gene under control of a promoter directing expression in a particular cell type, as a result of genetic engineering. Different promoters known to the skilled person may be suitable in the context of the present invention, such as, but not limited to neural-specific promoters or inducible promoters. Alternatively, such cells or organisms are selected from those naturally expressing the C10orf74 gene, such as, but not limited to brain, heart, placenta, kidney and skeletal muscle cells and/or cells naturally expressing the or the SCAMP5 gene, respectively. According to a particular embodiment such cells, naturally expressing the C10orf74 gene are neural cells, such as for instance cells from the cerrebellum or neurocampus. Optionally such cells are derived from an immortal cell line. According to a further particular embodiment such cells can be obtained from embryonic stem cells or from a neuronal cell line. Cells with aberrant expression of the C10orf74 or SCAMP5 gene can optionally also be obtained from individuals with a natural aberrant C10orf74 or SCAMP5 gene expression, respectively. Alternatively aberrant expression of the C10orf74 or SCAMP5 gene, more particularly as a result of an alteration in the C10orf74 or SCAMP5 gene, respectively, can be obtained by techniques known to the skilled person such as but not limited to site-directed mutagenesis or post-transcriptional gene silencing, e.g. by RNA interference that causes a decrease or loss of a functional gene product.

According to a particular embodiment of the present invention, aberrant expression of the C10orf74 and/or SCAMP5 gene is obtained in yeast. Preferably, according to the present invention, such an aberrant gene product, respectively corresponds to a C10orf74 gene product or a SCAMP5 product which has lost its original function.

The term “yeast cell” herein is used to mention unicellular fungi of the phylum Ascomycota that reproduce by fission or budding and are capable of fermenting carbohydrates into alcohol and carbon dioxide. Preferably said yeast cells are Saccharomycetaces or Schizossaccharomycetales, such as Saccharomyces pombe, Saccharomyces cerevisae, Candida or Pichia. Most preferably yeast cells of the species Saccharomyces cerevisae are used for manipulation in accordance with the present invention. A genetically stable yeast cell is also referred to as a yeast strain.

Thus, the present invention further provides a method of making a cell or non-human animal with absent, inappropriate or modified C10orf74 and/or SCAMP5 expression causing a decrease or loss of biological function in the gene(s) comprising either modifying a C10orf74 gene and/or a SCAMP5 gene, respectively, or the promoter thereof in the neural cell, wherein said modification is selected from a) substitution, b) deletion, c) frame-shift, d) insertion or ensuring post-transcriptional gene silencing by RNA interference or inhibition of the functional gene product; and thereafter selecting modified cells. Methods for modifying, more particularly inhibiting gene expression (e.g. knock-out) of a gene are known to the skilled person. According to a particular embodiment of the present invention, an engineered cell or is provided comprising a vector encoding inhibitory polynucleotide (e.g. such as for RNAi) specific for C10orf74 and/or SCAMP5 mRNA encoded by a heterologous gene relative to the genome of said cell. Also within the scope of the present invention A non-human animal is provided with loco-regional neural transgenes, wherein said animal comprises a vector encoding RNAi specific for C10orf74 and/or SCAMP5 mRNA encoded by a heterologous gene relative to the genome of said cell.

Specific hybridization when referring to a nucleic acid probe herein relates to the ability of the probe to form a stable hybrid only with the target molecule. More particularly, in the context of the present invention specific hybridization to a particular C10orf74 or SCAMP5 gene sequence relates to the ability of the probe to hybridize e.g. only to a particular alteration of the C10orf74 or SCAMP5 gene, respectively, (and not to the wild-type C10orf74 or SCAMP5 gene) or only to a particular wild-type C10orf74 or SCAMP5 gene (and not to e.g. sequences of with high similarity to C10orf74 or SCAMP5 gene family members, respectively).

Specific binding when referring to a ligand herein relates to the ability of the ligand to bind only to a specific target, e.g. polynucleotide. More specifically specific binding can relate to the fact that the ligand only binds to the wild-type C10orf74 or SCAMP5 gene product or to one or more aberrant forms of the C10orf74 or SCAMP5 gene product, respectively. Alternatively, specific binding can refer to the fact that the ligand will bind only the C10orf74 or SCAMP5 gene product, respectively and not the gene product of other genes, more particularly the gene product of other members of this gene family.

The following examples, not intended to limit the invention to specific embodiments described, may be understood in conjunction with the accompanying Figures, incorporated herein by reference, in which:

### LEGEND TO THE FIGURES

FIG. 1. Positional cloning of the breakpoint on chromosomes 14 and 16 in a patient with idiopathic autism. (A) Physical map of 14q12 (UCSC Genome Browser, July 2003 version—centromere on the left). BAC AL132827 and cosmid 85f1 were shown to span the breakpoint on chromosome 14. Arrows indicate the position of the translocation breakpoint with regard to the genomic clone (→, distal; ←, proximal). Also shown: the breakpoint position on 14q12 (a “red”), and the nearest genes to this breakpoint (amisyn, 300 kb upstream, and NOVA1, 1.1 Mb downstream). (B and C) FISH analysis on metaphase spread of the patient. (B) BAC AL0132827 (CTD-2149C7—left) and cosmid 85f1 (right) span the breakpoint on chromosome 14. (C) Hybridization with the subtelomere probe of 16q (green signal—left) showed the absence of subtelomere sequence on der14. Using the telomere probe (PNA—right), we showed the presence of an interstitial telomere on der16.

FIG. 2. Expression study of genes proximal to the breakpoint on chromosome 14q12. (A) Scheme of the chromosomal abnormality. The interstitial telomere on the der16 is shown in green, the neotelomere on the der14 is shown in blue. The G/A alleles of KIAA1305 and G/C alleles of amisyn are indicated on the respective chromosomes. (B) Expression analysis of amisyn and KIAA1305 in the patient. Sequence analysis showed heterozygosity for rs1052484 (G/C, amisyn) and rs8017377 (G/A, KIAA1305) in the patient. cDNA analysis revealed expression of the G allele only for amisyn, compared to biallelic expression in a control. Both G and A alleles are expressed for KIAA1305 in the patient mRNA.

FIG. 3. TRIP8 and C10orf74 genes affected by the inversion breakpoint at 10q21.3. (A) Physical map of 10q21.3 (UCSC Genome Browser, July 2003 version—centromere on the left). The three TRIP8 transcripts, and the NRBF-2 and C10orf74 genes are shown. The breakpoint position is indicated with a red bar. CpG islands in the region are shown, and the location of SNP rs3211105 in exon 20 of TRIP8 is indicated with an asterisk. (B) Expression analysis of TRIP8 in the patient. Sequence analysis showed heterozygosity for rs3211105 (T/C). Both T and C alleles are expressed in the patient mRNA, but the levels of the C allele are lowered. (C) RT-PCR in EBV cell lines of the patient and a control, as well as in human brain using transcript-specific primers (D) Expression analysis of NRBF-2 and C10orf74 in the patient (E) Methylation patterns of CpG37 proximal, and CpG122 and 125 distal to the 10q21.3 breakpoint in the patient (Pat) compared to control (Contr). Enzymes used are: BamHI (‘B’) and HpaII (‘H’) for CpG37, EcoRI (‘E’) and NotI (‘N’) for CpG122, and EcoRI (‘E’) and Cfr101 (‘C’) for CpG125.

FIG. 4. The PPDC-DC gene on 15q24.2 is disrupted, and position effect affects expression of the SCAMP5 gene. (A) Physical map of 15q24.2 (UCSC Genome Browser, July 2003 version—centromere on the left). The position of the BAC ends (AC125435 and AC113208) and cosmids used for FISH analysis are shown. Arrows indicate the position of the translocation breakpoint with regard to the genomic clone (→, distal; ←, proximal). (B) FISH analysis on metaphase spread of the patient shows that BAC AC015720 (left) and cosmid 32h10 span the breakpoint on chromosome 15. (C) Southern blot analysis with a probe from C32h10 reveals rearranged fragments (indicated by arrow) in the patient (P) compared to a control (C). (D) Expression analysis of SCAMP5 in the patient. Sequence analysis showed heterozygosity for rs8033925 (A/G) in the patient. cDNA analysis revealed expression of the G allele only, compared to biallelic expression in a control.

FIG. 5. Endogenous expression of Nbea, amisyn and SCAMP5 in neuroendocrine and neuronal mouse cell lines. (A) RT-PCR and (B) Western blot analysis. Cell lines used are α-TC1.6, β-TC3, AtT20 and Neuro2A (N2A). All three genes were shown to be expressed at a detectable level in β-TC3 cells.

FIG. 6. Western blot analysis showing the silencing capacity of the RNAi constructs (R) by means of transient co-transfection of HEK293T cells with a corresponding expression construct (E). (M) indicates the mock-transfected lanes. For each gene, the results of the mU6pro RNAi construct with the best silencing capacity is shown. For synaptotagmin IX, SytIX-R4 was shown to be the most efficient.

FIG. 7. Regulated secretion of Flag-Agrp in β-TC3 cells 3 days after co-transfection with RNAi (SCAMP5, Nbea and amisyn and YopI, CLIC4) or full-length expression construct (amisyn, SCAMP5, CLIC4). Number or experiments and standard deviations are indicated. p-values are p<0.0001 for Nbea, amisyn, SCAMP5 and SytIX, and p>0.05 for Clic4 and MALT1.

FIG. 8. Homo sapiens chromosome 10 open reading frame 74 (C10orf74), mRNA [SEQ ID NO:1]

FIG. 9. Homo sapiens amisyn mRNA, complete cds [SEQ ID NO:3]

FIG. 10. Homo sapients SCAMP5 mRNA, complete cds [SEQ ID NO:5]

### EXAMPLES

### Example 1

### Identification of Amisyn as a Gene Indicative of a NSD or a Predisposition Thereto

a) Primers and Probes

TABLE 1

Primers used to amplify and sequence DNA
and cDNA fragments of amisyn for SNP analysis.

| | | | |
|---|---|---|---|
| | | | SEQ |
| Nr | Primer | Location | ID |

| | | | |
|---|---|---|---|
| 1 | 5′-AAACTGCCTATCCTGGTGACTCTTC-3′ | amisyn ex6 | 7 |
| 2 | 5′-AAAATCTAAGACTGCTGTTTTTCCC-3′ | amisyn ex6 | 8 |
| 3 | 5′-AGAAAAATGCACCTTCTTCCAGATC-3′ | amisyn ex4 | 9 |
| 4 | 5′-AAAATCTAAGACTGCTGTTTTTCCC-3′ | amisyn ex6 | 10 |
| 5 | 5′-ATTAACTGCCAATCCAAAATTATGG-3′ | amisyn ex4 | 11 |
| 6 | 5′-TTTCCCAATAAATTCAATTGTTTTC-3′ | amisyn ex6 | 12 |
| 7 | 5′-TATTAAGCAGGAAGGCATTTTAATG-3′ | amisyn ex6 | 13 |
| 8 | 5′-TTTCCCAATAAATTCAATTGTTTTC-3′ | amisyn ex6 | 14 |

b) Case Report

R. R. is a twelve year old boy, referred for child psychiatric assessment for problems in social development, life-long severe emotional lability, poor development of play, fantasy and problem-solving, and primary encopresis. He is the second child of non-consanguineous parents. There was no family history of developmental disorders. Pregnancy and delivery at 42 weeks gestational age were uneventful. There were no signs of dysmaturity. He was born with a unilateral coloboma at the eye. Early milestones of psychomotor and language development were within normal range. R. R. was a very active toddler with poor development of symbolic play. Attention has always been a problem. Until primary school he remained more interested in sensopathic toys than age-appropriate games. Socializing with peers has always been a problem. R. R. has always been oversensitive for loud noises. He had problems to adjust to changes. Pediatric assessment of the encopresis did not show any somatic reasons for the problem. He had marked anxieties about toilets and the encopresis seemed a consequence of toilet avoidance. Clinical assessment showed a unilateral coloboma and no other dysmorphic sign. Social interactions lacked reciprocity and were characterized by interpretation problems, often leading to unexpected emotional reactions. Receptive and productive vocabulary and syntaxis were within normal range but there were marked impairments in language pragmatics, e.g. understanding figurative meaning, puns and irony. Non-verbal communication, e.g. emotion recognition, was impaired. Fast mood changes and anxiety were induced by often irrelevant triggers. R. R.'s attention was poor. He showed perseverations and was abnormally dependent on routines. Intelligence testing with Wechsler Intelligence Scale for Children Revised (WISC-R) showed a full scale IQ of 71, in the borderline range. Theory-of-mind testing with the Strange Stories Test showed marked deficits in Theory-of-Mind. According to DSM-IV criteria, R. R. fulfilled the criteria for pervasive developmental disorder and for primary encopresis.

c) Cytogenetic Analysis

In the patient, a de novo apparently balanced translocation was detected with karyotype 46,XY t(14;16)(q12;q24.3). In 22 out of 60 metaphases, loss of the small derivative chromosome 14 was observed. Therefore, this person has a mosaic monosomy for the proximal part of chromosome 14. Karyotype of the parents was normal.

d) Positional Cloning of the Breakpoint

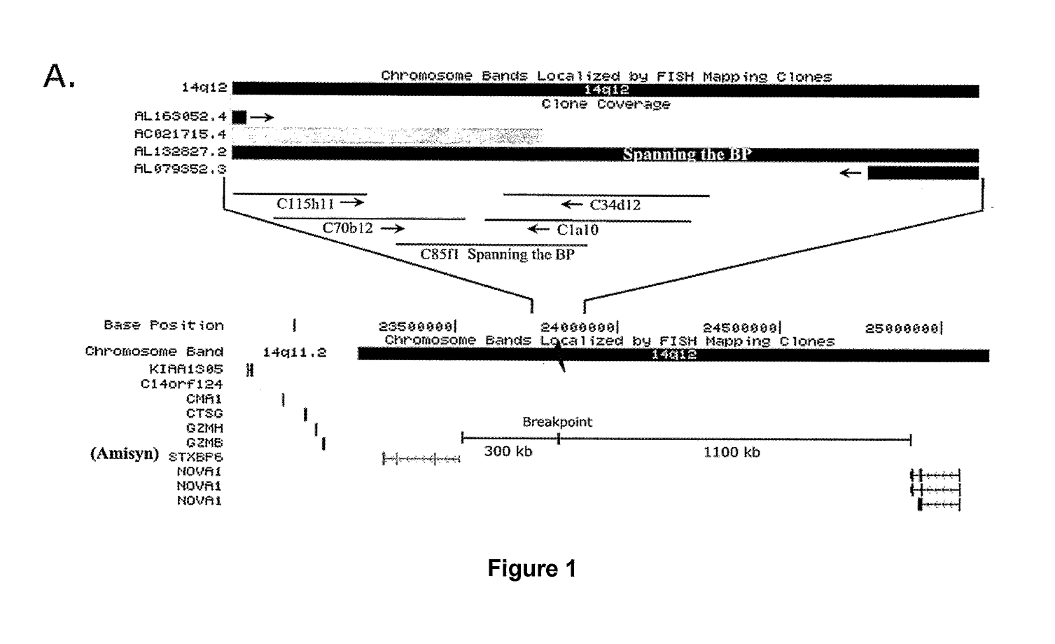

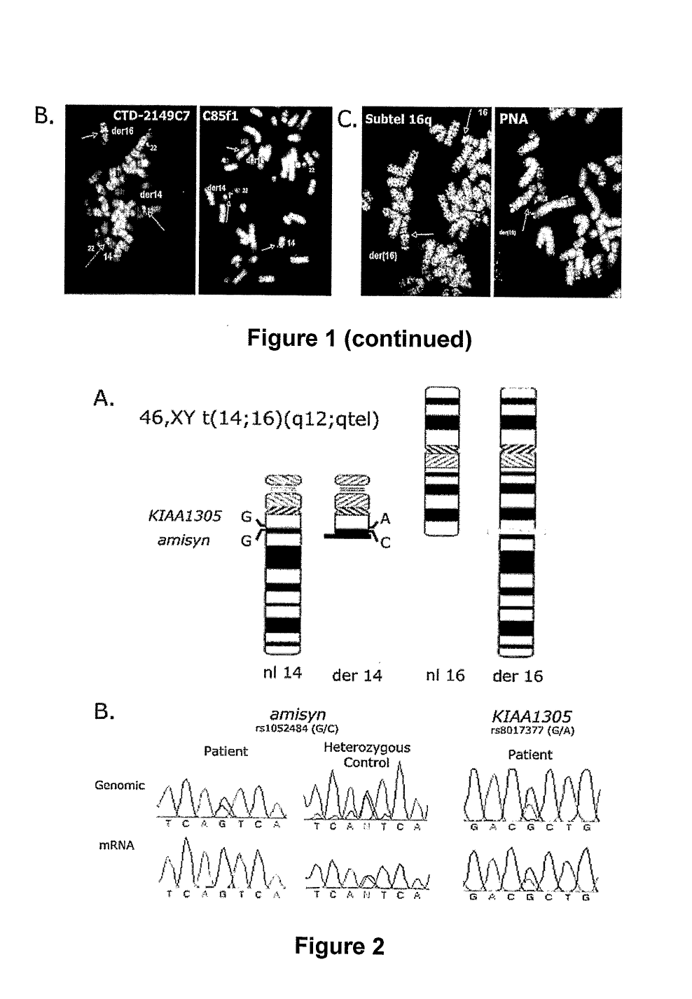

By means of FISH, it was shown that the subtelomeric region of the der16q appeared intact, with the presence of an interstitial telomere (FIG. 1C). Therefore, this constitutes a non-reciprocal translocation. As shown in FIG. 1A, the breakpoint on chromosome 14q12 was flanked by BACs RP11-769B21 (NCBI AL163052) and RP11-388G3 (NCBI AL079352), and BAC CTD-2149C7 (NCBI AL132827) was found to span the breakpoint (FIGS. 1A and B). In this BAC, the breakpoint was localized within cosmid 85f1 (FIGS. 1A and B). In silico analysis (Ensembl Genome Server, UCSC Genome Browser, NCBI-Entrez Genome—9 Jul. 2004) revealed that this region is devoid of known or predicted genes (FIG. 1A).

e) Amisyn Gene Expression is Disrupted By the Chromosomal Aberration on 14q12

Since no genes were found to be directly disrupted by the translocation, the expression of amisyn (NCBI NM_{—}014178), the gene closest to the breakpoint on 14q (300 kb more proximal, FIG. 1A), which appeared an attractive candidate gene was investigated. For one of the eight reported exonic SNPs, rs1052484 (in the 3′ UTR of amisyn), the patient was heterozygous for the G/C alleles (FIG. 1B). At the mRNA level, only the G-allele could be detected, indicating mono-allelic expression of the amisyn gene (FIG. 2B). These results were confirmed after subcloning of the different PCR products (data not shown). The possibility of mono-allelic expression due to genomic imprinting (Kotzot D. Clin Genet 2001;60(3):226-31) was excluded by studying four heterozygous controls, which showed biallelic expression of the amisyn gene in EBV cell lines (shown for one control, FIG. 2B). At the genomic level, one of the alleles (C) showed a lower signal, consistent with the mosaic loss of the derivative chromosome 14 (FIG. 2B). Interestingly, loss of amisyn expression was detected for this allele, confirming the loss of amisyn transcription from the rearranged chromosome (FIGS. 2A and B).

f) Amisyn, Evaluation as a Candidate Gene for Autism

Amisyn has recently been characterized as a syntaxin-binding protein (STXBP6) expressed mainly in brain tissue (Scales S J, et al. J Biol Chem 2002;277(31):28271-9). Therefore, Scales et al. named the protein amisyn (“ami” being French for “friend”), based on the naming of the related tomosyn protein (“tomo” meaning “friend” in Japanese).

Syntaxin belongs to one of the three distinct and well-conserved families of membrane-associated proteins, called SNAREs (soluble NSF [N-ethylmaleimide-sensitive fusion protein] attachment protein receptors) (Bennett M K, Scheller R H. Proc Natl Acad Sci U S A 1993;90(7):2559-63). Syntaxin is, like SNAP-25, referred to as a target membrane-associated SNAREs (t-SNAREs). In contrast, VAMP2 is called a vesicle-associated SNARE (v-SNARE). These v- and t-SNARE proteins play their role through the formation of so-called fusogenic SNARE complexes, leading to bilayer fusion (Bonifacino J S, Glick B S. Cell 2004;116(2):153-66). Since exocytosis requires the fusion of vesicular and plasma membranes, SNAREs play a crucial role in membrane fusion events, resulting in the release of vesicle contents into the extracellular space.

It is clear that a number of additional factors are required to regulate SNARE-mediated membrane fusion in vivo. These so-called SNARE regulators bind directly to the SNAREs and are involved in the regulation of SNARE assembly as well as the ability of SNAREs to participate in trafficking events (reviewed in Gerst J E. Biochim Biophys Acta 2003;1641 (2-3):99-110).

Amisyn contains a C-terminal VAMP-like coiled-coil domain, that binds specifically to t-SNAREs, like syntaxin-1 and to a lesser extent syntaxin-4 and SNAP25 (Scales et al., 2002, above). As a consequence, the coiled-coil domains of amisyn and VAMP2 are competitive in their binding to t-SNARE complexes (Scales et al, 2002, above). However, like tomosyn but unlike other SNAREs of the VAMP family, amisyn contains a unique N-terminus and lacks the hydrophobic stretch that could serve as a trans-membrane anchor (Scales et al., 2002, above). Therefore, amisyn cannot act as a classical v-SNARE. Since the syntaxin-1/SNAP25 complex plays an important role in vesicle fusion by its interaction with VAMP2, amisyn may be involved in regulating SNARE complex formation. Amisyn may act as a placeholder facilitating the binding of VAMP2 upon receipt of the appropriate stimulus. In addition, overexpression of the amisyn coiled-coil domain was found to inhibit secretion from cultured cells (Scales et al., 2002, above). This suggests that the coiled-coil domain of amisyn mimics the presence of VAMP v-SNARE and fulfils a “matchbreaker” role, preventing v-t SNARE assembly (Gerst J E, 2003, above). Very recently, a new family harbouring this type of proteins has been defined, the inhibitory class of SNAREs or i-SNAREs (Short B, Barr F A. Curr Biol 2004;14(5):R187-9).

In conclusion, amisyn may keep the t-SNARE complex in a conformation-inactive state and so governing the specificity as well as the timing of membrane fusion Scales et al., 2002, above).

### Example 2

### Identification of C10Orf74 as a Gene Indicative of a NSD or a Predisposition Thereto

a) Primers and Probes

TABLE 2

primers for the amplification of
the portions of C10orf74.

| | | |
|---|---|---|
| Primer | SEQ ID | Location |

| | | |
|---|---|---|
| 5′-AAACTGCCTATCCTGGTGACTCTTC-3′ | 15 | ex6 |
| 5′-AAAATCTAAGACTGCTGTTTTTCCC-3′ | 16 | ex6 |
| 5′-AGAAAAATGCACCTTCTTCCAGATC-3′ | 17 | ex4 |
| 5′-AAAATCTAAGACTGCTGTTTTTCCC-3′ | 18 | ex6 |
| 5′-ATTAACTGCCAATCCAAAATTATGG-3′ | 19 | ex4 |
| 5′-TTTCCCAATAAATTCAATTGTTTTC-3′ | 20 | ex6 |
| 5′-TATTAAGCAGGAAGGCATTTTAATG-3′ | 21 | exon 6 |
| 5′-TTTCCCAATAAATTCAATTGTTTTC-3′ | 22 | exon 6 |
| 5′-CACTGACATTGGCAACTTCCTGAAG-3′ | 23 | exon 9 |
| 5′-AACACTGACGCCAGCGAAGAGT-3′ | 24 | exon 9 |
| 5′-ATTGTCACCAATGAGCAGATTCACATCCTG-3′ | 25 | exon 7 |
| 5′-GAATCTCTTCATGGTGCCTGATGACCCC-3′ | 26 | exon 9 |
| 5′-GAATCTCTTCATGGTGCCTGATGACCCC-3′ | 27 | exon 8 |
| 5′-ATCATGGAGGCTGAGGCACCGCA-3′ | 28 | exon 9 |

TABLE 3
sequencing primers for C100RF174

| | |
|---|---|
| Primer | Location |

| | |
|---|---|
| 5′-CAAAGACAGTATCATCTGAAGTTGCCTAATAAGG-3′ | C10orf74 |
| (SEQ ID:29) | intr7 |
| 5′-ATTTCAATTCAAAGCATTGTCTTATAAAAAGCAG-3′ | Behind |
| (SEQ ID:30) | C10orf74 |
| 5′-TCAAGAACTTTATCTGTGGGAGCTGATTTGCACC-3′ | C10orf74 |
| (SEQ ID:31) | intr7 |
| 5′-TTTTTTCAGAACAGTGAACGATTTAAAGGGAGAC-3′ | Behind |
| (SEQ ID:32) | C10orf74 |
| 5′-GTTGGATTCCATGAATTGGTATTAC-3′ | C10orf74 |
| (SEQ ID:33) | ex8 |
| 5′-ATTTCTCTCTAAAGCTTAAATGTGAGG-3′ | C10orf74 |
| (SEQ ID:34) | ex8 |
| 5′-ACTAAAACACTGAAAAAAAATGCCG-3′ | C10orf74 |
| (SEQ ID:35) | ex8 |

TABLE 4

RT-PCR primers used for
C10orf74 cDNA amplification.

| | | SEQ |
|---|---|---|
| Name | Primer | ID |
| C10orf74 | 5′-TTGCCAATGCAAAATACAGTTGTCATTAATCC-3′ | 36 |
| up | | |
| C10orf74 | 5′-AACATCTACCAAAATAGTTCCCTAAATAGGTTTGT | 37 |
| low | GC-3′ | |

b) Case Report

C. K. is a nine year old boy, referred for child psychiatric assessment because of social and communication problems. He is the third child of non-consanguineous parents. There was no family history of developmental disorders. Pregnancy and delivery were uneventful. There were no signs of dysmaturity. Psychomotor milestones were within normal range. Early language and social development were delayed. Development of fantasy and pretend play were delayed and as a preschooler, C. K. was fascinated by circling objects. He did not socialize with peers at school. Clinical assessment showed a boy with no dysmorphic signs. Clinical neurological examination was normal. He had marked deficits in social reciprocity, and “active-but-odd” type social interactions. Productive and receptive language were mildly delayed. Non-verbal communication was impaired. He showed perseverative behaviour and had problems to adjust to changes. Mood and affect were normal. There were signs of a formal thought disorder. In a special school program, C. K. had one year delay in reading and arithmetics. Wechsler's intelligence scales (WISC-revised) showed an uneven intelligence profile within the normal range: performal IQ 106, verbal IQ 93. Theory-of-Mind testing showed marked impairment in social perspective taking. In conclusion, C. K. fulfilled sufficient criteria for the diagnosis of (high-functioning) autistic disorder according to DSM-IV. His mild formal thought disorder is not a typical characteristic in autism.

c) Cytogenetic Analysis

In the patient, an apparently balanced inversion was detected with karyotype 46,XY inv(10)(q11.1;q21.3). Karyotype of the parents was normal.

d) Positional Cloning of the Breakpoints

The positions of the breakpoints on chromosome 10q were determined by means of FISH on metaphase chromosomes of the patient.

The distal breakpoint at 10q21.3 was flanked by BACs RP11-44419 (NCBI AL607128) and RP11-439F7 (NCBI AL607062). BAC RP11-351O1 (NCBI AC022022) and cosmids 87d1 , 14h8 and 29h9 were found to span the breakpoint. Southern blot analysis with a probe from the overlap region of these cosmids showed a rearranged fragment in the DNA of the patient using the EcoRI restriction enzyme. The breakpoint could thus be localised to a 7.3 kb EcoRI restriction fragment. Sequence analysis of the cosmid ends and of the probe used in the Southern blot study, and comparison with the human genome sequence, showed that the 10q21.3 breakpoint is located inside the first intron of the TRIP8 gene (Katoh M. Int J Mol Med 2003;12(5):817-21).

Although cytogenetically the breakpoint was mapped at 10q11.1, the centromere probe of chromosome 10 used as a control probe for FISH gave a split signal on the rearranged chromosome 10 . This revealed that the proximal breakpoint was located in the repetitive sequence of the centromere of chromosome 10.

e) Breakpoint Position Effect: C10orf74 Expression is Affected By the Inversion Breakpoint at 10q21.3

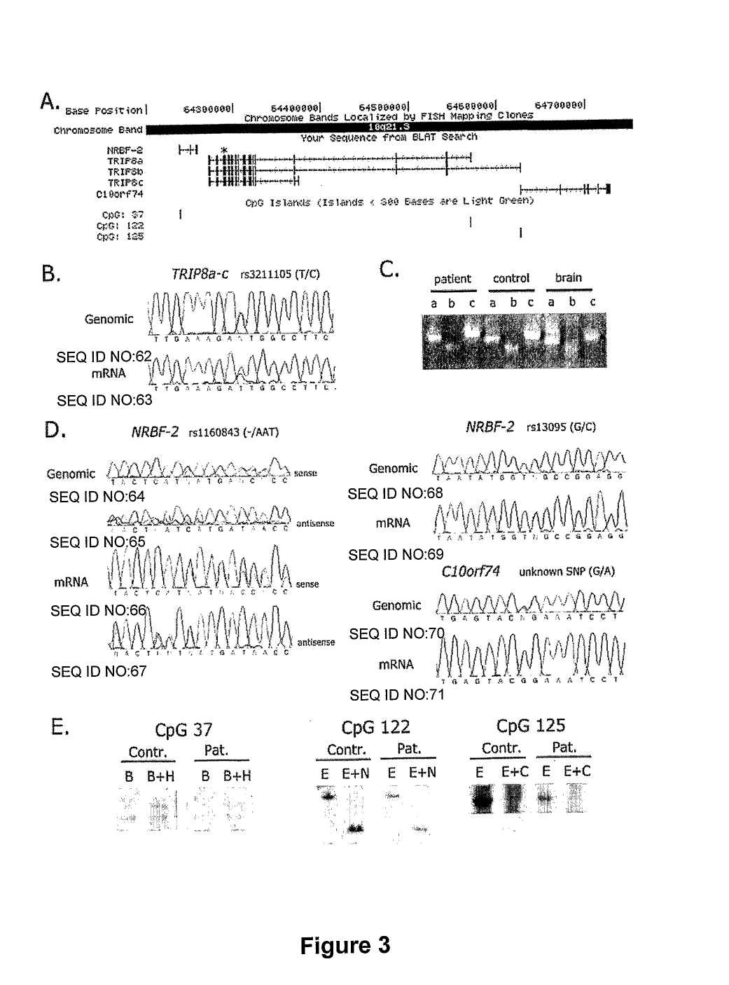

The proximal breakpoint was located in the repetitive sequence of the centromere of chromosome 10. As a consequence, 10q21.3 sequences are now juxtaposed to centromere sequences in this patient, and position effects may have occurred in genes more distant from this breakpoint. Therefore, we investigated the expression levels of genes and the methylation pattern of CpG islands both proximal and distal to the breakpoint (FIG. 3A).

Using SNPs, we investigated the expression of the genes NRBF-2 [NM_{—}030759] proximal, and C10orf74 [NM_{—}001001330] distal to the breakpoint (FIG. 3D). For the predicted gene C10orf74 (NCBI mRNA BC068557 and BC018628), the patient was found to be heterozygous for an unreported SNP (G/A) at the 3′ UTR. At the mRNA level only the G-allele could be detected, indicating mono-allelic expression of this gene (FIG. 3D). For two of the five reported exonic SNPs in the 3′UTR of NRBF-2 (NCBI mRNA AK054957), rs1160843 (−/AAT) and rs13095 (G/C), the patient was heterozygous. Analysis of the corresponding mRNA fragments revealed a normal biallelic expression of this NRBF-2 gene (FIG. 3D). As shown in FIG. 3E, the methylation of the investigated methylation-dependent restriction sites at or nearby CpG island 37 proximal, and islands 122 and 125 distal to the breakpoint in the patient were unaffected by the vicinity of the centromere sequence as compared to control. RT-PCR in EBV cell lines of the patient and a control, as well as in human brain using transcript-specific primers revealed expression of TRIP8a and TRIP8c, but low levels of TRIP8b. (FIG. 3C). Sequence analysis showed heterozygosity for rs1160843 (−/AAT) and rs13095 (G/C) in NRBF-2, and a G/A SNP in C10orf74 in the patient (FIG. 3D). For both polymorphisms in NRBF-2, both alleles are expressed in the patient mRNA. cDNA analysis of the G/A SNP in C10orf74 revealed expression of the G allele only for this gene.

f) C10orf74 Gene: Genomic Structure, Homologs in Other Species and Protein Family

Initially, the database predicted C10orf74 (mRNA BC068557) and BC018658 (mRNA BC018658) as two separate genes. The unreported G/A SNP was located in the BC018658 gene. However, the distance between the last exon of gene C10orf74 and the first (and only) exon of BC018658 was 1.2 kb, suggesting that both may represent one single gene. This was confirmed by the finding of a cDNA clone (IMAGE562868 [NCBI ESTs AA100605 and AA100609]) containing part of the 3′ end of C10orf74 and the entire BC018658 mRNA. In addition, this was proven by RT-PCR, with sense primer in BC068557 and antisense primer in BC018658, and subsequent sequencing of both this amplified cDNA fragment and the IMAGE562868 clone (data not shown). This transcript was also found on the AceView database, referred to as TB2_DP1_HVA22.4b.

In conclusion, the full-length C10orf74 gene (NM_{—}001001330) consists of 8 exons spanning 104 kb of the human genome at 10q21.3. The gene codes for a transcript of 4677 bp, with a coding sequence of 765 bp (255 AA).

Through BLASTP search, proteins homologous to the predicted protein corresponding to human C10Orf74 (MH68557) in other species, like mouse (AAH04607, D10UCla1 gene [NM_{—}178606]), rat (XP_{—}215383, LOC294375 gene [XM_{—}215383]), zebrafish (AAH45373, MGC55529 gene [NM_{—}200161]), Drosphila (MM68228, CG30193 gene [NM_{—}166571]), C. elegans (AAC46595, T19C3.4 gene [NM_{—}064820]), and S. cerevisiae (CAA07720, Yop1p gene [AJ007902]) were identified. For mouse, rat and zebrafish, we were able to show syntheny between human 10q23.1 and the chromosomal region where the homolous gene was located (chr10 in mouse, chr20 in rat, and chr12 in zebrafish). Therefore, the corresponding proteins are true orthologs of the human C10orf74 (see FIG. 11).

The yeast Yop1p gene has several homologs in human, of which C5orf18 [NM_{—}005669] at 5q22.2 coding for TB2/DP1 (AAH65926) is likely to be the true ortholog (Calero M, Whittaker G R, Collins R N. J Biol Chem 2001;276(15):12100-12). Alignment of protein sequences of the orthologs and homologs of C10Orf74, revealed a conserved N-terminal region, corresponding to the “TB2/DP1 and HVA22” domain (Pfam PF03134, FIG. 11).

g) Evaluation of C10orf74 as Candidate Genes for Autism

Functional data of the C10Orf74 are available on two homologs, the yeast Yop1p and a plant homolog HVA22.

Yop1p (Yip one partner) was identified as a probable regulator of cellular vesicle trafficking, since it interacts with Ypt1p (Yang X, et al. Embo J 1998;17(17):4954-63). (a Rab GTPase required for transport from the endoplasmatic reticulum (ER) to the Golgi complex; Segev N, et al. Cell 1988;52(6):915-24), as well as with Yip1p (Calero et al., 2001, above) (a Ypt1p-interacting protein; Yang X. et al., 1998, above) and Yif1p (Ito T et al. Proc Natl Acad Sci U S A 2001 ;98(8):4569-74) (a Yip1p interacting factor; Matern Het al. Embo J 2000;19(17):4485-92; Barrowman Jet al. J Biol Chem 2003;278(22):19878-84). It is likely that Yop1p (also called Yip2p, Yang X et al., 2002, above), Yip1p and Yif1p, together with several other Yip family members (Shakoori A, et al. Biochem Biophys Res Commun 2003;312(3):850-7), form an integral membrane protein complex binding to Ypt1p. It is thought that these ‘Rab effector’ complexes serve as cores recruiting different ‘effector partner’ proteins involved in membrane trafficking to their proper subcellular localization (e.g. synaptic vesicle VAMP2 interacts with PRA1/Yip3p (Martincic I et al. J Biol Chem 1997;272(43):26991-8), and ER-to-Golgi SNAREs Bos1p and Sec22 interact with Yif1p-Yip1p; Barrowman Jet al. J Biol Chem 2003;278(22):19878-84). However, besides this putative role as Rab effector, Rab-interaction might also indicate a function for Yop as a ‘Rab regulator’ (e.g. Yip3 as a GDP dissociation inhibitor (GDI) displacement factor (Sivars U et al. Nature 2003;425(6960):856-9).

Overexpression of Yop1p blocks membrane trafficking, resulting in huge swollen cells of aberrant shape (Calero et al., 2001, above). This block was shown to be at the level of the ER, leading to an accumulation of internal membrane structures (Calero et al., 2001, above). Interestingly, this phenotype could be rescued by co-expression of Yip1p, further indicating that Yop1p and Yip1p function in a common transport step (Calero et al., 2001, above). Similarly, mutant cells depleted of Yip1p and Yip1p thermosensitive mutations massively accumulate ER membranes and display aberrations in protein secretion (Yang X et al., 2002, above). This suggests an essential role of Yip1p in recruiting Rab GTPase complexes to Golgi target membranes in preparation for fusion. On the other hand, anti-Yip1p antibodies did not inhibit vesicle tethering or fusion when added after vesicle production (Barrowman Jet al. J Biol Chem 2003;278(22):19878-84) or preincubation of ER membranes with coat protein COPII (Heidtman M et al. J Cell Biol 2003;163(1):57-69), suggesting an early requirement for Yip1p in vesicle formation. In one possible scenario, Yip1p-Yif1p might cluster cargoes, increasing the likelihood for vesicle formation at a specific point (Spang A. Curr Biol 2004;14(1):R33-4). Consistent with the findings concerning Yop1p and Yip1p overexpression, a potential role for Yop1p can be suggested as a negative regulator of Yip1p function in vesicle biogenesis, severing the diffusion limitation set by Yip1p-Yif1p once enough cargo has been assembled (Spang A. Curr Biol 2004;14(1):R33-4). In addition, it is possible that Yip1p serves as a regulatory checkpoint in vesicle budding to ensure that ER-derived vesicles can ultimately interact with Rab GTPases (Heidtman M et al. J Cell Biol 2003;163(1):57-69). In such a model, the Rab protein may not be required per se for vesicle budding, but the Rab-binding activity would be required.

Also, in yeast, an interaction partner was identified (called SEY1 for synthetic enhancement of Yop1; Brands A, Ho T H. Plant Physiol 2002;130(3):1121-31), which is a homolog of the Arabidopsis RHD3 (root hair defective 3; Galway M E, Heckman J W, Jr., Schiefelbein J W. Planta 1997;201(2):209-18). Recently, RHD3 was shown to play its role in membrane transport between the ER and the Golgi apparatus (Zheng H, Kunst L, Hawes C, et al. Plant J 2004;37(3):398-414). In addition, the yeast double mutant of Yop1p and Sey1 is defective in vesicular trafficking, resulting in an accumulation of transport vesicles and a decreased secretion (Brands et al., 2002, above). The authors suggest that HVA22/Yop1p play a role to facilitate membrane turnover, or to decrease unnecessary secretion.

Expression of the HVA22 plant gene is induced by environmental stresses, such as dehydration, salinity and extreme temperatures, and by abscisic acid stress hormone (Shen Q. et al. J Biol Chem 1993;268(31):23652-60). However, the role of the HVA22 protein in stress response is not yet understood. Based on observations about the yeast Yop1p homolog, it has been suggested that HVA22 regulates vesicular traffick in stressed cells, either to facilitate membrane turnover, or to decrease unnecessary secretion (Brands et al., 2002, above).

### Example 3

### Identification of SCAMP5 as a Gene Indicative of a NSD or a Predisposition Thereto.

a) Primers and Probes Used

TABLE 5
primers used for SCAMPS amplification

| | |
|---|---|
| Primer | SEQ ID |

| | |
|---|---|
| 5′-tggccgtgaacctggtgg-3′ | 38 |
| 5′-cttccccagggaccaatcattac-3′ | 39 |

TABLE 6
nested primers used for cDNA amplification

| | |
|---|---|
| Primer | SEQ ID |

| | |
|---|---|
| 5′-ctgctcctacgtctgctggtttc-3′ | 40 |
| 5′-agctccccatcagcttccttg-3′ | 41 |

TABLE 7

primers used for qDNA amplification,
gDNA and cDNA sequencing.

| | |
|---|---|
| Primer | SEQ ID |

| | |
|---|---|
| 5′-cccttcccttttctccttcccta-3′ | 42 |
| 5′-actcagccttggggaagacaaag-3′ | 43 |

b) Case Report

R. R. is a twelve year old boy, referred for child psychiatric assessment for problems in social development, life-long severe emotional lability, poor development of play, fantasy and problem-solving, and primary encopresis. He is the second child of non-consanguineous parents. There was no family history of developmental disorders. Pregnancy and delivery at 42 weeks gestational age were uneventful. There were no signs of dysmaturity. He was born with a unilateral coloboma at the eye. Early milestones of psychomotor and language development were within normal range. R. R. was a very active toddler with poor development of symbolic play. Attention has always been a problem. Until primary school he remained more interested in sensopathic toys than age-appropriate games. Socializing with peers has always been a problem. R. R. has always been oversensitive for loud noises. He had problems to adjust to changes. Pediatric assessment of the encopresis did not show any somatic reasons for the problem. He had marked anxieties about toilets and the encopresis seemed a consequence of toilet avoidance. Clinical assessment showed a unilateral coloboma and no other dysmorphic sign. Social interactions lacked reciprocity and were characterized by interpretation problems, often leading to unexpected emotional reactions. Receptive and productive vocabulary and syntaxis were within normal range but there were marked impairments in language pragmatics, e.g. understanding figurative meaning, puns and irony. Non-verbal communication, e.g. emotion recognition, was impaired. Fast mood changes and anxiety were induced by often irrelevant triggers. R. R.'s attention was poor. He showed perseverations and was abnormally dependent on routines. Intelligence testing with Wechsler Intelligence Scale for Children Revised (WISC-R) showed a full scale IQ of 71, in the borderline range. Theory-of-mind testing with the Strange Stories Test showed marked deficits in Theory-of-Mind. According to DSM-IV criteria, R. R. fulfilled the criteria for pervasive developmental disorder and for primary encopresis.

c) Cytogenic Analysis

In the patient, a balanced translocation with involvement of the chromosomes 1 and 15 was identified. From the karyotyping of the patient, it seemed that the translocation probably is in or near the cytogenetic band 1p35.3. For the first FISH analysis, YAC clones laying in the flanking cytogenetic bands were used. The YAC clones 828E9 (contains STS marker D1S2702, in the band 1p36.12) and 750G5 (contains STS marker D1S201, in the band 1p35.1) are found telomeric and centromeric to the breakpoint respectively. Subsequently, BAC's and PAC's were selected in the region between those 2 YAC clones and analysed by FISH. The BAC's/PAC clones from the contigs NT004610 and NT032979 are found telomeric to the breakpoint, while those from the contigs NT004782, NT004538 and NT004511 are located centromeric to the breakpoint. The contig NT004391 spans the breakpoint. The BAC clones laying in the latter contig were subsequently analysed by FISH and the BAC RP11-108J9 (AL662924) appeared to span the breakpoint.

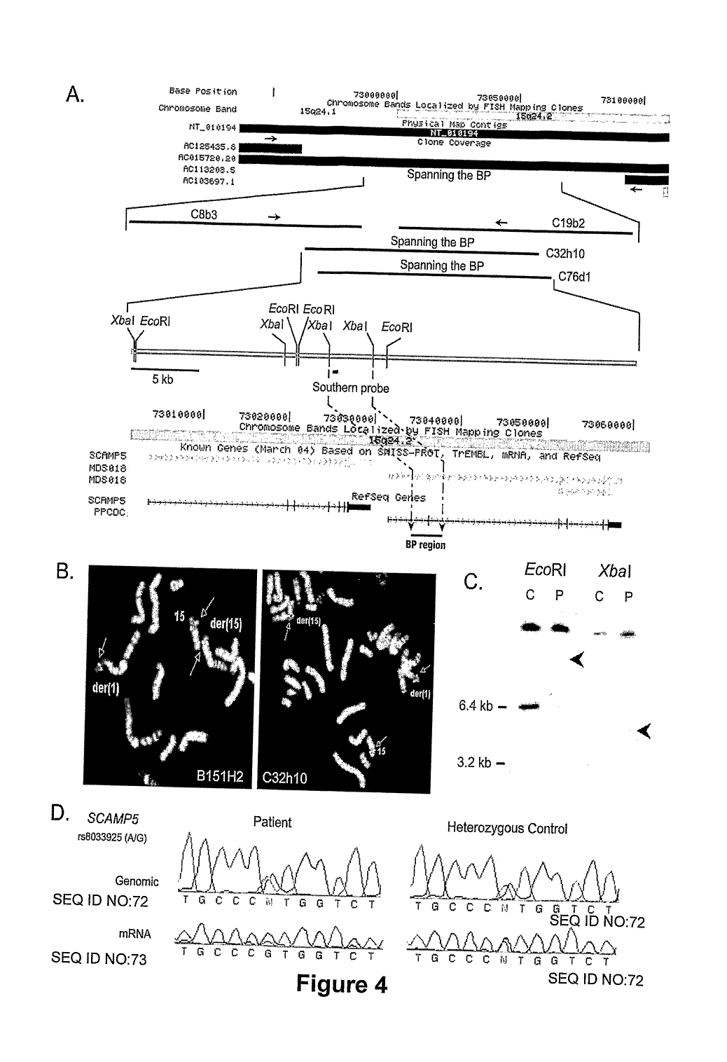

d) Translocation Effect on Genes In and Proximal to the Breakpoint (FIG. 4)

Although the break on chromosome 15q24.2 was shown to be located in the flavoprotein-coding gene PPDC-DC (NCBI NM_{—}021823), the expression of SCAMP5 (NCBI NM_{—}138967), the gene 10 kb more proximal to the breakpoint, was investigated. For exonic SNP, rs8033925 (in the 3′ UTR of SCAMP5), the patient was heterozygous for the A and G alleles. Analysis of the corresponding mRNA fragments in blood platelets of the patient revealed a biallelic expression of this SCAMP5 gene. However, the expression from the A allele was significantly reduced compared to two heterozygous controls, which showed clear normal biallelic expression of the SCAMP5 gene in their blood platelets. In conclusion, the SCAMP5 gene is still expressed from both alleles, however, the expression from the A allele is markedly affected by the chromosomal aberration.

e) Evaluation of SCAMP5 as Candidate Gene for Autism

SCAMP5 is the brain-specific member of the secretory carrier membrane protein family involved in vesicle budding from trans-Golgi. In general, SCAMPs are composed of a cytoplasmic N-terminal sequence with NPF repeats, four central transmembrane regions (TMRs) and a cytoplasmic tail. In many proteins NPF repeats are binding sites for EH (Eps15 homology) domain proteins, which are involved in clathrin-mediated vesicle budding from the plasma membrane or trans-Golgi complex.

In addition, SCAMPs have a conserved basic aromatic segment between TMR 2 and 3, facing the cytosol, called the E-peptide. It has been reported that the E-peptide of SCAMP2 binds to membranes containing PI(4,5)P₂ and Na⁺,K⁺/H⁺ exchanger NHE7 protein. Moreover, overexpression of both the E-peptide itself and full-length SCAMP2 with a mutated E-peptide result in a reduced exocytosis. Interestingly, in contrast to SCAMPs 1-3, SCAMP4 and 5 both lack the N-terminus containing the NPF repeats. We suggest that, as a consequence, these proteins play a role as negative regulators of exocytosis/regulated secretion.

### Example 4

### Endogenous Expression of Genes Involved in Regulated Secretion in Mouse Cell Lines

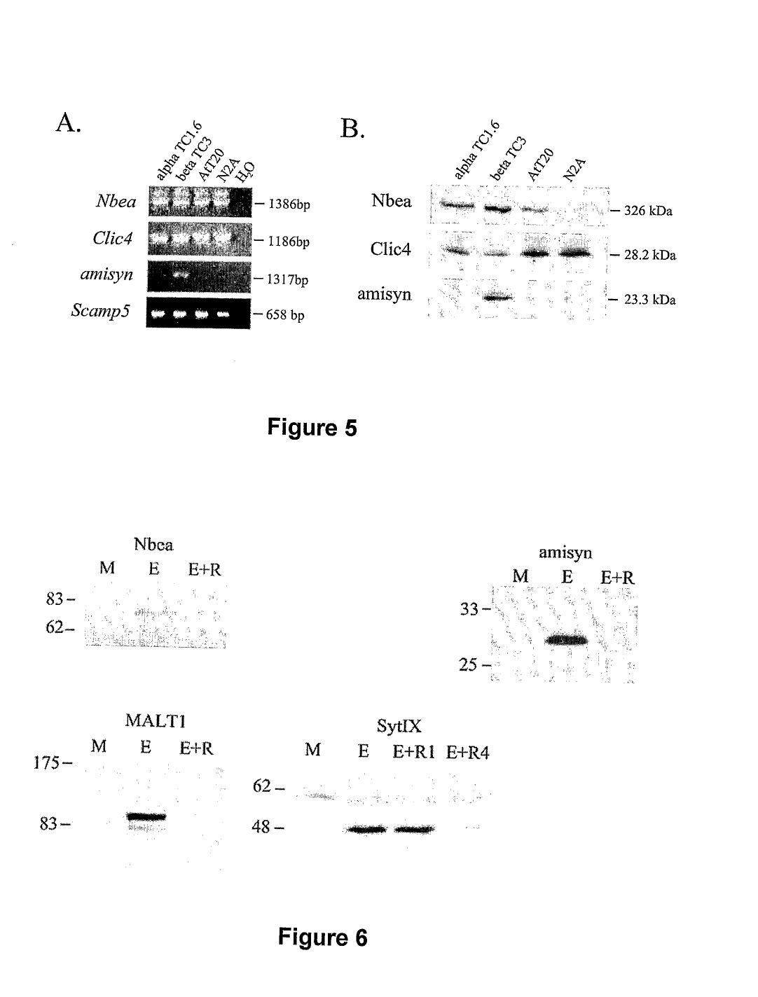

By means of RT-PCR and Western blot analysis, the endogenous expression of Nbea, amisyn and SCAMP5 in both neuroendocrine (α-TC1.6 and β-TC3 cells) and neuronal (AtT20 and Neuro2A cells) mouse cell lines was investigated (FIG. 5, A). Nbea and SCAMP5 were found to be expressed in all four cell lines. In contrast, amisyn showed only expression in the neuroendocrine β-TC3 cell line.

By means of RT-PCR, it was determined that synaptotagmin IX, used as a control for the RSA, is also expressed in β-TC3 cells. In addition, the C10orf74 gene was found to be expressed in β-TC3 cells (data not shown).

Since neuroendocrine cells contain LDCVs but not SSVs, the β-TC3 cell line forms an excellent model system to identify compounds which modulate the secretion of LDCVs.

### Example 5

### Effect of Aberrant Gene Expression on Vesicle Transport

The role of neurobeachin (NBEA), SCAMP5 and amisyn in regulated secretion is demonstrated by way of gene silencing by transient expression of small interference RNAs (siRNAs). inhibition of Synaptogagmin, a protein known to be involved in regulated secretion was used as a positive control.

Regulated secretion of large dense-core vesicles was studied by the secretagogues 3-isobutyl-1-methylxanthine (IBMX) and forskolin using Agouti related protein (Agrp) as marker proteins.

RNAi Constructs

The 19-mer target regions (see Table 8) were selected using Ambions' siRNA Target Finder (http)://www.ambion.com/techlib/misc/siRNA finder.html). Further selection of the targets was based on following criteria: (1) for use of the mU6pro vector, the target sequence should start with a G, (2) since a 4-6 nucleotide poly(T) acts as a termination signal for RNA pol III, avoid stretches of ≧4 T's or A's, (3) sequences having 30-50% GC content were found to be most active, and (4) to guarantee gene-specific knockdown, eliminate any target sequence with more than 15 contiguous base pairs (bp) of homology to other coding sequences. For each of these genes, several target sequences were selected at different positions along the length of the gene.

TABLE 8

Target sequences used for RNAi-mediated
knockdown of genes in the regulated
secretion assay.

| | | |
|---|---|---|
| Gene | Target sequence | SEQ ID |

| | | |
|---|---|---|
| neurobeachin | 5′-GGAACTAATTCCAGAGTTC-3′ | 44 |
| amisyn | 5′-GGCGAATACTTAACTTATA-3′ | 45 |
| MALT1 | 5′-GTTTCGCTCACCATGCTTC-3′ | 46 |
| synaptotagmin | 5′-AATGGATGTAGGAGGACTC-3′ | 47 |
| IX-R1 | | |
| synaptotagmin | 5′-GAATGAGGCCATCGGGAGG-3′ | 48 |
| IX-R4 | | |
| SCAMP5 | 5′-gctcttagcatggttcata-3′ | 49 |

Upper and lower 60-mer (Eurogentec) encoding the shRNAs, were designed using pSilencer Converter (http://www.ambion.com/techlib/misc/psilencer_converter.html). The upper oligonucleotide is composed of the 19-mer sense target sequence, a loop sequence (TTCAAGAGA) and the antisense target sequence, followed by a poly(T) 6-mer (RNA polymerase III Terminator) and a GGAA. The lower oligonucleotide is the complement of the described sense oligonucleotide, except for the 5′- and 3′-ends which are designed to create the overhang necessary for directional cloning into the vector: 5′-TTT-3′ at the 5′ end of the upper primer and 5′-GATC-3′ at the 5′ end of the lower primer. Remark: at the 3′ end of the lower primer, the C of the antisense strand corresponding to the first G of the target sequence is left out, resulting in an actual 5′ overhang of TTTG (BbsI).

After annealing of the 60-mers (5 nmol of each oligo in 50 μl annealing buffer in boiling water slowly cooling down), this double-stranded fragment was ligated (T4 ligase, Promega) into the mU6pro vector digested with BbsI and BamHI. We used a mU6pro vector in which the sequence encoding a GFP-tag has been deleted (NcoI/EcoRI digestion and Klenow treatment, followed by blunt ligation). The mU6pro RNAi construct silencing the human ‘Mucosa-associated Lymphoid tissue Translocation protein 1’ gene (MALTI) was kindly provided by Dr. Thijs Baens, Departement Menselijke Erfelijkheid, University of Leuven.

Expression Constructs

In order to obtain expression constructs for Nbea, SCAMP5, amisyn and synaptotagmin IX (SytIX), PCR was performed on cDNA obtained by RT-PCR of total RNA from mouse embryonic brain. In addition, a human MALTI construct (Flag-pcDNA3.1) expressing the full-length coding sequence for use as control was provided by Dr. Thijs Baens.

Constructs are instrumental in the evaluation of the efficiency of the corresponding siRNAs by co-transfection of 293T cells, as well as in overexpression experiments. Primers used are shown in Table 9.

TABLE 9

Cloning strategy of mouse
expression constructs.

| | | | | |
|---|---|---|---|---|
| Gene | Nr | Fragment | Primers | bp |

| | | | | |
|---|---|---|---|---|
| Nbea | 1* | 1863-2566 | 5′-CCCCAATGCCAGAAGATTCA | 2115 |
| | | AA | G-3′ | |
| | | | (SEQ ID NO: 50 ) | |
| | | | 5′-GTGGGCTCGTGGAAGGAAA | |
| | | | C-3′ | |
| | | | (SEQ ID NO: 51) | |
| amisyn | 3** | Full-length | 5′-CTGCCATCAGCAAGGAAATTT | 633 |
| | | CDS | TTG-3′ | |
| | | | (SEQ ID NO: 52) | |
| | | | 5′-ACTCCTCCACTTTCTTTAGAG | |
| | | | CTTCACC-3′ | |
| | | | (SEQ ID NO: 53) | |
| SytIX | 4* | Full-length | 5′-GGTCTCCAGCGCCTAAGACAC | 1169 |
| | | CDS | CTCCAG-3′ | |
| | | | (SEQ ID NO: 54) | |
| | | | 5′-GGGATCTGGGGTCAGGTGCTT | |
| | | | GCTTG-3′ | |
| | | | (SEQ ID NO: 55) | |
| Scamp | | AA2-236 | 5′-gcggagaaagtgaacaacttc | 714 |
| 5 | | full-length | c-3′ | |
| | | CDS | (SEQ ID NO: 56) | |
| | | | (at 5′additional HA- | |
| | | | tag) | |
| | | | 5′-cttggctggctcacatctc-3 | |
| | | | SEQ ID NO: 57 | |

All constructs contain the entire coding sequence with exception of construct 1. The sequences of the primers used to amplify the respective fragments are indicated, as are the length of the PCR fragment and the restriction sites used for insertion in the expression vector.
* and ** = constructs used to test silencing efficiency of the RNAi target sequences, and
** = constructs used for overexpression experiments.

In order to clone PCR products into the pcDNA3.1 HisA expression vector (Invitrogen), primers were preceded by the appropriate 12-mer oligo's, 5′-GACTACGGATCC-3′ (SEQ ID NO:58 ) for BamHI and 5′-GATCAGGAATTC-3′ (SEQ ID NO: 59 ) for EcoRI. PCR fragments were purified (QIAquick gel extraction kit, Qiagen), followed by a digestion and another purification step (QIAquick PCR purification kit, Qiagen). For SCAMP5 cloning into pcDNA3 expresion vector, primers were preceded by 5′-GACTACGGATCC-3′ (SEQ ID NO: 60) for BamHI and 5′-GATCAGCTCGAG-3′ (SEQ ID NO: 61) for XhoI. Primary antibodies used for detection of the different expression constructs are anti-Xpress (Invitrogen) for constructs 1-4 and anti-Flag M2 (Sigma-Aldrich) for MALT1 construct. Secondary antibodies used are horseradish peroxidase-linked: Rabbit anti-Mouse (DAKO) for tagged constructs, and Swine anti-Rabbit (DAKO) for endogenous proteins.

Regulated Secretion Assay

The assay (based on the assay as described in Creemers et al. J Biol Chem 1996;271(41):25284-91) is performed using cells after transient co-transfection (Lipofectamine 2000) of a C-terminal Flag-tagged Agrp construct with the mU6pro RNAi construct or an overexpression construct (described above). Agrp is a soluble protein that is efficiently sorted to the regulated secretory pathway.

Mouse β-TC3 cells were transfected in 6-well plates (BD Biosciences). After 2 days the cells were washed and incubated for 18 h with serum free medium to reduce the tonic release of stored material induced by serum factors. Subsequently, the cells were incubated for 3h in serum free DMEM, followed by a 3 h incubation in the presence of secretagogues that stimulate large dense-core vesicles secretion. Secretoagogues used are 10 μM forskolin (Sigma), an adenylate cyclase activator, and 0.1 mM of the phosphodiesterase inhibitor IBMX (Sigma). Medium was collected (A, absence and B, presence of secretagogues), and cells were lysed in 1× Sample Buffer (fraction C). Proteins of fractions A and B were precipitated. Proteins of fractions A-C were blotted and detected using anti-Flag M2 as primary and Rabbit anti-Mouse as secondary antibody (see above).

The signal was quantified using Kodak Digital Science (Kodak Imager with ID Image Analysis Software, version 3.0).

Finally, the relative amounts of regulated secretion ZN is calculated:

$${Z}_{N} = \frac{{[ {Y}_{+} - {Y}_{-} ]}_{N}}{{[ {Y}_{+} - {Y}_{-} ]}_{C}} = \frac{{[ ( {X}_{+} / {X}_{L} ) - ( {X}_{-} / {X}_{L} ) ]}_{N}}{{[ ( {X}_{+} / {X}_{L} ) - {X}_{-} / {X}_{L} ]}_{C}}$$

With X=intensity of sum of band per lane
X_{L}=intensity of cell lysate
X₋=intensity of medium without secretagogues
X₊=intensity of medium with secretagogues

Thus, Y=intensity X normalysed to the lysate per lane
Y_{L}=1
Y₋=X₋/X_{L}=constitutive secretion
Y₊=X₊/X_{L}=constitutive and regulated secretion [symbol] Y_{R}=Y₊−Y₋=regulated secretion

and, Z=regulated secretion Y_{R} for lane N normalysed to the control lane C (without RNAi or expression construct). Therefore, Z_{C}=1
Z_{N}=Z for lane N with RNAi or expression construct

Results

Silencing Capacity of the Different RNAi Constructs

To study the silencing capacity of the RNAi constructs, their efficiency to suppress expression from a recombinant construct representing the gene of interest was investigated by co-transfection of HEK293T cells with both RNAi and expression constructs. As shown in FIG. 6 for Nbea and amysin, a very efficient shRNA construct was obtained for each gene.

For Nbea, suppression of endogenous protein was shown by means of immuno-fluorescence analysis, performed three days after co-transfection of AtT20 cells with Nbea-RNAi and LPP-GFP constructs. In contrast, earlier, i.e. two days after transfection, Nbea was still detectable in transfected cells. This was probably due to the long half-life of the protein produced before RNAi-mediated gene silencing has completely suppressed expression.

Regulated Secretion Assay

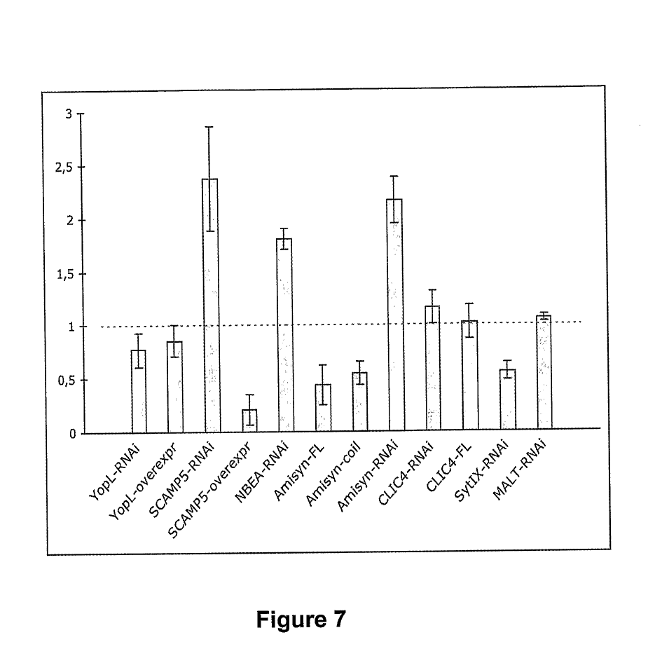

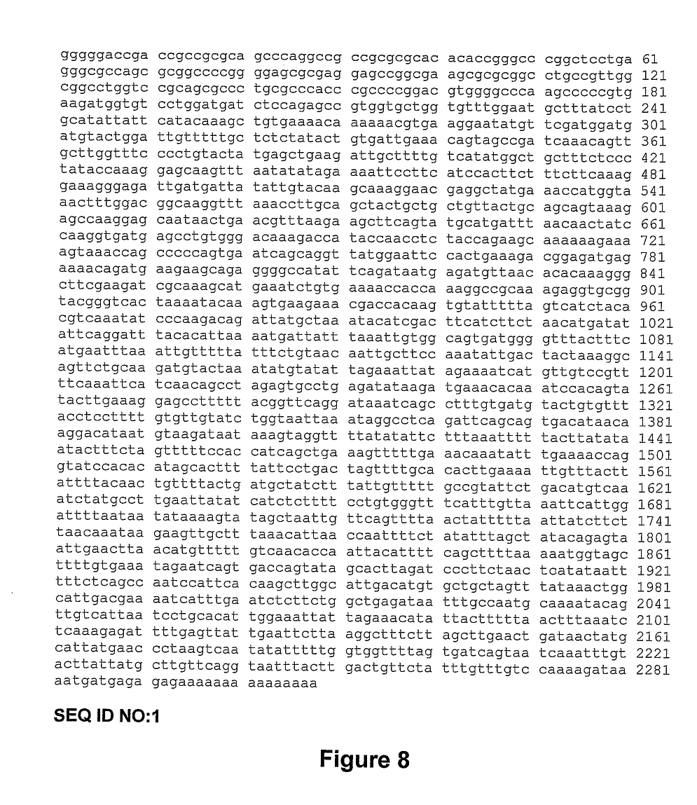

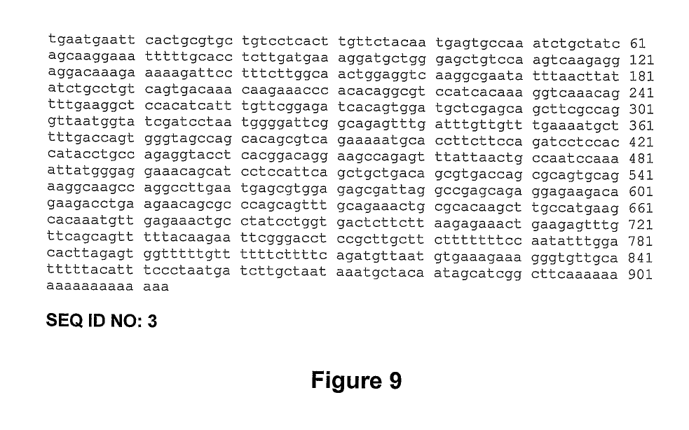

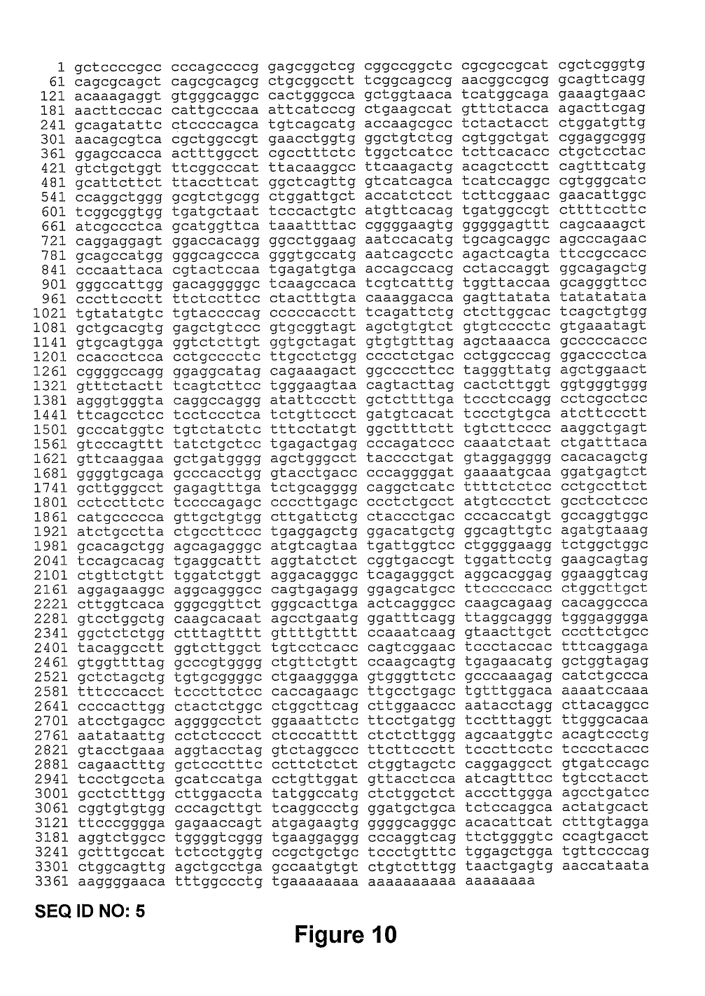

As can be seen in FIG. 7, inhibition of Nbea, and amisyn and SCAMP5 using RNAi results in a significant increase in regulated secretion of the Flag-Agrp in beta-TC3 cells. Interestingly, overexpression of full-length amisyn and SCAMP5 result in a significant decrease of regulated secretion. We were not able to show any significant involvement for CLIC4 in the regulated secretion of LDCVs, confirming that this is not a negative regulator of secretion.

In conclusion, these data suggest a role for NBEA, amisyn and SCAMP5 proteins as negative regulators of neuron vesicle trafficking and/or fusion. Moreover, it implies that vesicle trafficking in neurons is involved in the pathogenesis of autism.

1. A method for identifying a patient which has been diagnosed with a neural system disorder as susceptible to the treatment with a medicament capable of influencing targeted secretion, said method comprising detecting, in a biological sample of said patient, aberrant expression of one or more genes encoding proteins involved in regulated secretion.

2. The method according to claim 1, which comprises detecting aberrant regulated secretion in cells of said patient.

3. The method of claim 1, which comprises detecting increased regulated secretion in isolated cell samples of said patient.

4. The method of claim 3, wherein said cells are hematopoietic cells or blood cells.

5. The method of claim 4, wherein said cells are platelets.

6. The method of claim 3, wherein said cells are neural cells.

7. The method according to claim 1, which comprises detecting aberrant expression levels of said genes and/or the expression of aberrant gene products in a biological sample of said animal.

8. The method of claim 1 or 2, said method comprising detecting the presence of an alteration in one or more genes involved in regulated secretion.

9. The method of claim 8, wherein said alteration is a chromosomal alteration or a sequence alteration selected from the group consisting of a translocation, an inversion, a deletion, an insertion or a substitution.

10. The method of claim 9, wherein said alteration results in a reduction or loss of function of said one or more genes.

11. The method of any one of claims 7 to 10, wherein detection of aberrant gene expression is achieved by detecting altered expression levels of the said one or more genes.

12. The method of any one of claims 7 to 11, which comprises detecting aberrant gene expression of genes encoding negative regulators of regulated secretion.

13. The method of claim 12, which comprises detecting aberrant gene expression of at least two genes encoding negative regulators of regulated secretion.

14. The method of claim 12, which comprises detecting aberrant gene expression of at least two genes selected from the group consisting of Neurobeachin, amisyn, c10orf74 and SCAMP5.

15. The method of claim 12, which comprises detecting aberrant gene expression of the C10orf74 gene and/or of the SCAMP5 gene.

16. A method of testing or screening an animal for a neural system disorder or a predisposition to a neural system disorder, said method comprising detecting for at least two genes involved in regulated secretion whether there is aberrant expression; whereby aberrant expression of at least one of said genes is indicative of a neural system disorder or a predisposition thereto.

17. The method according to claim 16, which comprises detecting aberrant expression levels of said genes and/or the expression of aberrant gene products in a biological sample of said animal.

18. The method of claim 16 or 17, said method comprising detecting for said at least two genes involved in regulated secretion, whether or not there is an alteration in said genes.

19. The method of claim 18, wherein said alteration is a chromosomal alteration or a sequence alteration selected from the group consisting of a translocation, an inversion, a deletion, an insertion or a substitution.

20. The method of claim 19, which comprises determining for said at least two genes involved in regulated secretion whether said alteration results in a reduction or loss of function of said genes.

21. The method of any one of claims 16 to 20, wherein detection of aberrant gene expression is achieved by detecting altered expression levels of the said one or more genes.

22. The method of any one of claims 7 to 21, wherein detection of aberrant expression is achieved by detecting altered levels of the mRNA transcripts or mRNA precursor.

23. The method of any one of claims 7 to 22, which comprises (A) extraction of the chromosomal material from said sample, (B) amplification of the chromosomal material using PCR; (C) optionally, sequencing said material; and (D) determining the presence of an alteration in said nucleotide sequence.

24. The method of any one of claims 11 or 20, wherein said loss of function results in an increase in regulated secretion.

25. The method of any one of claims 7 to 24, wherein said at least two genes are involved in the secretion of large core dense vesicles.

26. The method of any one of claims 7 to 25, wherein said at least two genes are selected from the group consisting of NBEA, c10orf74, SCAMP5 and amisyn.

27. The method of claim 7 or 12, which comprises detecting the altered expression of the gene products of said one or more genes using specific ligands.

28. The method of claim 27, wherein said altered expression of said gene products is detected using labelled ligands to said gene product.

29. The method of claim 28, wherein the said ligands are polyclonal antibodies.

30. The method of claim 28, wherein the said ligands are monoclonal antibodies.

31. The method of any of the claims 1 to 30, characterised in that the neural system disorder is autism.

32. A method of screening for a therapeutic agents for use in the prevention and/or treatment of a neural system disorder, said method comprising: (A) providing an isolated cell sample comprising one or more genes involved in regulated secretion (B) introducing to the cell a agent to be screened; and (C) determining whether said agent influences said regulated secretion;

33. The method of claim 32, wherein the expression of said one or more genes involved in regulated secretion is modified or the normal functioning of the gene product of said gene is inhibited.

34. The method of claim 33, wherein the expression of said gene is modified using antisense, RNAi, homologous recombination or transposons.

35. The method of claim 33, wherein said one or more genes are wild-type genes.

36. The method of claim 33, wherein said one or more genes are functionally altered versions of wild-type genes.

37. The method of claim 33, wherein said one or more genes are exogenous to said cell.

38. The method of claim 37, wherein said one or more genes are heterologous to said cell.

39. The method of claim 37 or 38, wherein said one or more exogenous genes are wild-type genes.

40. The method of claim 37 or 38, wherein said one or more exogenous genes are functionally altered versions of wild-type genes.

41. The method of any one of claims 33 to 40, wherein said modification of said one ore more genes results in increased regulated secretion.

42. The method of any one of claims 32 to 41, wherein said genes are selected from the group consisting of tomosyn, amisyn, C10orf74, SCAMP5 and neurobeachin.

43. A method of testing or screening an animal for a neural system disorder or a predisposition to a neural system disorder, said method comprising detecting aberrant expression of C10orf74 and/or amisyn and/or SCAMP5.

44. The method of claim 43, said method comprising detecting the presence of an alteration in the C10orf74 and/or amisyn and/or SCAMP5 gene.

45. The method of claim 44, wherein said alteration of the C10Orf74 and/or amisyn gene is a chromosomal alteration or a sequence alteration selected from the group consisting of a translocation, an inversion, a deletion, an insertion or a substitution.

46. The method of claim 44 or 45, wherein said alteration in the C10orf74 and/or amisyn gene is detected by hybridisation with a labelled probe.

47. The method of any one of claims 43 to 46, wherein detection of aberrant C10Orf74 and/or amisyn gene expression is achieved by detecting altered expression levels of the C10orf74 and/or amisyn gene, respectively.

48. The method of any one of claim 43 to 47, wherein detection of aberrant C10Orf74 and/or amisyn gene expression is achieved by detecting altered levels of the mRNA transcripts or mRNA precursors.

49. The method of any one of claims 43 to 47, which comprises (A) extraction of the chromosomal material from said sample, (B) amplification of the chromosomal material using PCR; (C) optionally, sequencing said material; and (D) determining the presence of an alteration in said nucleotide sequence.

50. The method of claim 43, which comprises detecting the altered expression of the C10orf74 and/or amisyn and/or SCAMP5 gene product using specific ligands.

51. The method of claim 50, wherein said altered expression of the C10Orf74 and/or amisyn and/or SCAMP5 gene product is detected using labelled ligands to said gene product.

52. Use of a polynucleotide sequence of the wild-type C10orF74 and/or amisyn and/or SCAMP5 gene or a variant C10Orf74 and/or amisyn and/or SCAMP5 gene for the diagnosis of a neural system disorder or the predisposition to a neural system disorder in an animal based on a biological sample of said animal.

53. The use according to claim 52, for the manufacture of a kit for the identification of individuals having a neural system disorder or a predisposition to a neural system disorder.

54. An isolated C10Orf74 and/or amisyn polynucleotide characterized in that said sequence includes at least one alteration of the C10orf74 and/or amisyn and/or SCAMP5 gene respectively, wherein said alteration results in aberrant expression of the C10orf74 and/or amisyn and/or SCAMP5 gene, respectively and is selected from the group consisting of a) a substitution, b) a deletion, d) an insertion, or e) one of a chromosomal inversion, a translocation or deletion.

55. An isolated cell containing the polynucleotide of claim 54.

56. A method of screening for a therapeutic agents for use in the treatment or therapy of a neural system disorder comprising: (A) providing an engineered yeast cell, comprising an introduced nucleotide sequence comprising C10orf74 and/or amisyn and/or SCAMP5 gene or an allelic variant, minigene, a synthetic gene or a homologue thereof; (B) introducing to the cell a compound, chemical signal or agent to be screened; and (C) correlating the change in said cell with the activity of the compound, chemical signal or agent.

57. A kit for use in the identification of a patient diagnosed with an autistic spectrum disorder as a patient susceptible to the treatment with a modulator of regulated secretion, characterized in that it comprises one or more probes which hybridize specifically with one or more genes encodng proteins involved regulated expression or variants thereof.

58. The kit of claim 57, wherein said genes are selected from neurobeachin, amisyn, SCAMP5 and C10orf74.

59. Use of an inhibitor of regulated secretion in the manufacture of a medicament for the treatment of patients which have been diagnosed with autism spectrum disorder and having aberrant expression of genes involved in regulated secretion.


SEQUENCE LISTING (73 sequences)

SEQ ID NO: 1 - DNA, 2308 bp, Homo sapiens
  CDS (184)..(951): C10orf74
gggggaccgaccgccgcgcagcccaggccgccgcgcgcacacaccgggcccggctcctgagggcgccagcgcggccccggggagcgcgaggagccggcgaagcgcgcggcctgccgttggcggcctggtccgcagcgccctgcgcccacccgccccggacgtggggcccaagcccccgtgaagatggtgtcctggatgatctccagagccgtggtgctggtgtttggaatgctttatcctgcatattattcatacaaagctgtgaaaacaaaaaacgtgaaggaatatgttcgatggatgatgtactggattgtttttgctctctatactgtgattgaaacagtagccgatcaaacagttgcttggtttcccctgtactatgagctgaagattgcttttgtcatatggctgctttctccctataccaaaggagcaagtttaatatatagaaaattccttcatccacttctttcttcaaaggaaagggagattgatgattatattgtacaagcaaaggaacgaggctatgaaaccatggtaaactttggacggcaaggtttaaaccttgcagctactgctgctgttactgcagcagtaaagagccaaggagcaataactgaacgtttaagaagcttcagtatgcatgatttaacaactatccaaggtgatgagcctgtgggacaaagaccataccaacctctaccagaagcaaaaaagaaaagtaaaccagcccccagtgaatcagcaggttatggaattccactgaaagacggagatgagaaaacagatgaagaagcagaggggccatattcagataatgagatgttaacacacaaagggcttcgaagatcgcaaagcatgaaatctgtgaaaaccaccaaaggccgcaaagaggtgcggtacgggtcactaaaatacaaagtgaagaaacgaccacaagtgtatttttagtcatctacacgtcaaatatcccaagacagattatgctaaatacatcgacttcatcttctaacatgatatattcaggatttacacattaaaatgattatttaaattgtggcagtgatggggtttactttcatgaatttaaattgtttttatttctgtaacaattgcttccaaatattgactactaaaggcagttctgcaagatgtactaaatatgtatattagaaattatagaaaatcatgttgtccgttttcaaattcatcaacagcctagagtgcctgagatataagatgaaacacaaatccacagtatacttgaaaggagcctttttacggttcaggataaatcagcctttgtgatgtactgtgtttacctccttttgtgttgtatctggtaattaaataggcctcagattcagcagtgacataacaaggacataatgtaagataataaagtaggttttatatattctttaaatttttacttatataatactttctagtttttccaccatcagctgaaagtttttgaaacaaatatttgaaaaccaggtatccacacatagcacttttattcctgactagttttgcacacttgaaaattgtttacttattttacaactgttttactgatgctatctttattgtttttgccgtattctgacatgtcaaatctatgccttgaattatatcatctcttttcctgtgggtttcatttgttaaattcattggattttaataatataaaagtatagctaattgttcagttttaactatttttaattatcttcttaacaaataagaagttgctttaaacattaaccaattttctatatttagctatacagagtaattgaacttaacatgtttttgtcaacaccaattacattttcagcttttaaaaatggtagcttttgtgaaatagaatcagtgaccagtatagcacttagatcccttctaactcatataatttttctcagccaatccattcacaagcttggcattgacatgtgctgctagtttataaactggcattgacgaaaatcatttgaatctcttctggctgagataatttgccaatgcaaaatacagttgtcattaatcctgcacattggaaattattagaaacatattactttttaactttaaatctcaaagagattttgagttattgaattcttaaggctttcttagcttgaactgataactatgcattatgaaccctaagtcaatatatttttggtggttttagtgatcagtaatcaaatttgtacttattatgcttgttcaggtaatttacttgactgttctatttgtttgtccaaaagataaaatgatgagagagaaaaaaaaaaaaaaa

SEQ ID NO: 2 - protein, 255 aa, Homo sapiens
MVSWMISRAVVLVFGMLYPAYYSYKAVKTKNVKEYVRWMMYWIVFALYTVIETVADQTVAWFPLYYELKIAFVIWLLSPYTKGASLIYRKFLHPLLSSKEREIDDYIVQAKERGYETMVNFGRQGLNLAATAAVTAAVKSQGAITERLRSFSMHDLTTIQGDEPVGQRPYQPLPEAKKKSKPAPSESAGYGIPLKDGDEKTDEEAEGPYSDNEMLTHKGLRRSQSMKSVKTTKGRKEVRYGSLKYKVKKRPQVYF

SEQ ID NO: 3 - DNA, 913 bp, Homo sapiens
  CDS (4)..(672): Amisyn
tgaatgaattcactgcgtgctgtcctcacttgttctacaatgagtgccaaatctgctatcagcaaggaaatttttgcacctcttgatgaaaggatgctgggagctgtccaagtcaagaggaggacaaagaaaaagattcctttcttggcaactggaggtcaaggcgaatatttaacttatatctgcctgtcagtgacaaacaagaaacccacacaggcgtccatcacaaaggtcaaacagtttgaaggctccacatcatttgttcggagatcacagtggatgctcgagcagcttcgccaggttaatggtatcgatcctaatggggattcggcagagtttgatttgttgtttgaaaatgcttttgaccagtgggtagccagcacagcgtcagaaaaatgcaccttcttccagatcctccaccatacctgccagaggtacctcacggacaggaagccagagtttattaactgccaatccaaaattatgggaggaaacagcatcctccattcagctgctgacagcgtgaccagcgcagtgcagaaggcaagccaggccttgaatgagcgtggagagcgattaggccgagcagaggagaagacagaagacctgaagaacagcgcccagcagtttgcagaaactgcgcacaagcttgccatgaagcacaaatgttgagaaactgcctatcctggtgactcttcttaagagaaactgaagagtttgttcagcagtttttacaagaattcgggacctccgcttgcttctttttttccaatatttggacacttagagtggtttttgttttttcttttcagatgttaatgtgaaagaaagggtgttgcatttttacatttccctaatgatcttgctaataaatgctacaatagcatcggcttcaaaaaaaaaaaaaaaaaaa

SEQ ID NO: 4 - protein, 222 aa, Homo sapiens
MNSLRAVLTCSTMSAKSAISKEIFAPLDERMLGAVQVKRRTKKKIPFLATGGQGEYLTYICLSVTNKKPTQASITKVKQFEGSTSFVRRSQWMLEQLRQVNGIDPNGDSAEFDLLFENAFDQWVASTASEKCTFFQILHHTCQRYLTDRKPEFINCQSKIMGGNSILHSAADSVTSAVQKASQALNERGERLGRAEEKTEDLKNSAQQFAETAHKLAMKHKC

SEQ ID NO: 5 - DNA, 3408 bp, Homo sapiens
  CDS (163)..(870): Scamp5
gctccccgcccccagccccggagcggctcgcggccggctccgcgccgcatcgctcgggtgcagcgcagctcagcgcagcgctgcggcctttcggcagccgaacggccgcggcagttcaggacaaagaggtgtgggcaggccactgggccagctggtaacatcatggcagagaaagtgaacaacttcccaccattgcccaaattcatcccgctgaagccatgtttctaccaagacttcgaggcagatattcctccccagcatgtcagcatgaccaagcgcctctactacctctggatgttgaacagcgtcacgctggccgtgaacctggtgggctgtctcgcgtggctgatcggaggcgggggagccaccaactttggcctcgcctttctctggctcatcctcttcacaccctgctcctacgtctgctggtttcggcccatttacaaggccttcaagactgacagctccttcagtttcatggcattcttctttaccttcatggctcagttggtcatcagcatcatccaggccgtgggcatcccaggctggggcgtctgcggctggattgctaccatctccttcttcggaacgaacattggctcggcggtggtgatgctaattcccactgtcatgttcacagtgatggccgtcttttccttcatcgccctcagcatggttcataaattttaccggggaagtggggggagtttcagcaaagctcaggaggagtggaccacaggggcctggaagaatccacatgtgcagcaggcagcccagaacgcagccatgggggcagcccagggtgccatgaatcagcctcagactcagtattccgccacccccaattacacgtactccaatgagatgtgaaccagccacgcctaccaggtggcagagctggggccattgggacagggggctcaagccacatcgtcatttgtggttaccaagcagggttcccccttcccttttctccttccctactttgtacaaaggaccagagttatatatatatatatatgtatatgtctgtaccccagcccccacctttcagattctgctcttggcactcagctgtgggctgcacgtggagctgtcccgtgcggtagtagctgtgtctgtgtcccctcgtgaaatagtgtgcagtggaggtctcttgtggtgctagatgtgtgtttagagctaaaccagcccccacccccaccctccacctgcccctcttgcctctggcccctctgaccctggcccagggacccctcacggggccaggggaggcatagcagaaagactggccccttcctagggttatgagctggaactgtttctactttcagtcttcctgggaagtaacagtacttagcactcttggtggtgggtgggagggtgggtacaggccagggatattcccttgctcttttgatccctccaggcctcgcctccttcagcctcctcctccctcatctgttccctgatgtcacattccctgtgcaatcttcccttgcccatggtctgtctatctctttcctatgtggcttttctttgtcttccccaaggctgagtgtcccagttttatctgctcctgagactgagcccagatccccaaatctaatctgatttacagttcaaggaagctgatggggagctgggccttacccctgatgtaggaggggcacacagctgggggtgcagagcccacctgggtacctgacccccaggggatgaaaatgcaaggatgagtctgcttgggcctgagagtttgatctgcaggggcaggctcatcttttctctcccctgccttctcctccttctctccccagagcccccttgagcccctctgcctatgtccctctgcctcctccccatgcccccagttgctgtggcttgattctgctaccctgaccccaccatgtgccaggtggcatctgccttactgccttccctgaggagctgggacatgctgggcagttgtcagatgtaaaggcacagctggagcagagggcatgtcagtaatgattggtccctggggaaggtctggctggctccagcacagtgaggcatttaggtatctctcggtgaccgttggattcctggaagcagtagctgttctgtttggatctggtaggacagggctcagagggctaggcacggagggaaggtcagaggagaaggcaggcagggcccagtgagaggggagcatgccttcccccaccctggcttgctcttggtcacagggcggttctgggcacttgaactcagggcccaagcagaagcacaggcccagtcctggctgcaagcacaatagcctgaatgggatttcaggttaggcagggtgggaggggaggctctctggctttagttttgttttgttttccaaatcaaggtaacttgctcccttctgcctacaggccttggtcttggcttgtcctcacccagtcggaactccctaccactttcaggagagtggttttaggcccgtggggctgttctgttccaagcagtgtgagaacatggctggtagaggctctagctgtgtgcggggcctgaaggggagtgggttctcgcccaaagagcatctgcccatttcccaccttcccttctcccaccagaagcttgcctgagctgtttggacaaaaatccaaaccccacttggctactctggcctggcttcagcttggaacccaatacctaggcttacaggccatcctgagccaggggcctctggaaattctcttcctgatggtcctttaggtttgggcacaaaatataattgcctctcccctctcccattttctctcttgggagcaatggtcacagtccctggtacctgaaaaggtacctaggtctaggcccttcttccctttcccttcctctcccctaccccagaactttggctccctttcccttctctctctggtagctccaggaggcctgtgatccagctccctgcctagcatccatgacctgttggatgttacctccaatcagtttcctgtcctacctgcctctttggcttggacctatatggccatgctctggctctacccttgggaagcctgatcccggtgtgtggcccagcttgttcaggccctgggatgctgcatctccaggcaactatgcactttcccggggagagaaccagtatgagaagtgggggcagggcacacattcatctttgtaggaaggtctggcctggggtcgggtgaaggagggcccaggtcagttctggggtcccagtgacctgctttgccattctcctggtgccgctgctgctccctgtttctggagctggatgttccccagctggcagttgagctgcctgagccaatgtgtctgtctttggtaactgagtgaaccataataaaggggaacatttggccctgtgaaaaaaaaaaaaaaaaaaaaaaaaaa

SEQ ID NO: 6 - protein, 235 aa, Homo sapiens
MAEKVNNFPPLPKFIPLKPCFYQDFEADIPPQHVSMTKRLYYLWMLNSVTLAVNLVGCLAWLIGGGGATNFGLAFLWLILFTPCSYVCWFRPIYKAFKTDSSFSFMAFFFTFMAQLVISIIQAVGIPGWGVCGWIATISFFGTNIGSAVVMLIPTVMFTVMAVFSFIALSMVHKFYRGSGGSFSKAQEEWTTGAWKNPHVQQAAQNAAMGAAQGAMNQPQTQYSATPNYTYSNEM

SEQ ID NO: 7 - DNA, 25 bp, artificial sequence
  primer
aaactgcctatcctggtgactcttc

SEQ ID NO: 8 - DNA, 25 bp, artificial sequence
  primer
aaaatctaagactgctgtttttccc

SEQ ID NO: 9 - DNA, 25 bp, artificial sequence
  primer
agaaaaatgcaccttcttccagatc

SEQ ID NO: 10 - DNA, 25 bp, artificial sequence
  primer
aaaatctaagactgctgtttttccc

SEQ ID NO: 11 - DNA, 25 bp, artificial sequence
  primer
attaactgccaatccaaaattatgg

SEQ ID NO: 12 - DNA, 25 bp, artificial sequence
  primer
tttcccaataaattcaattgttttc

SEQ ID NO: 13 - DNA, 25 bp, artificial sequence
  primer
tattaagcaggaaggcattttaatg

SEQ ID NO: 14 - DNA, 25 bp, artificial sequence
  primer
tttcccaataaattcaattgttttc

SEQ ID NO: 15 - DNA, 25 bp, artificial sequence
  primer
aaactgcctatcctggtgactcttc

SEQ ID NO: 16 - DNA, 25 bp, artificial sequence
  primer
aaaatctaagactgctgtttttccc

SEQ ID NO: 17 - DNA, 25 bp, artificial sequence
  primer
agaaaaatgcaccttcttccagatc

SEQ ID NO: 18 - DNA, 25 bp, artificial sequence
  primer
aaaatctaagactgctgtttttccc

SEQ ID NO: 19 - DNA, 25 bp, artificial sequence
  primer
attaactgccaatccaaaattatgg

SEQ ID NO: 20 - DNA, 25 bp, artificial sequence
  primer
tttcccaataaattcaattgttttc

SEQ ID NO: 21 - DNA, 25 bp, artificial sequence
  primer
tattaagcaggaaggcattttaatg

SEQ ID NO: 22 - DNA, 25 bp, artificial sequence
  primer
tttcccaataaattcaattgttttc

SEQ ID NO: 23 - DNA, 25 bp, artificial sequence
  primer
cactgacattggcaacttcctgaag

SEQ ID NO: 24 - DNA, 22 bp, artificial sequence
  primer
aacactgacgccagcgaagagt

SEQ ID NO: 25 - DNA, 30 bp, artificial sequence
  primer
attgtcaccaatgagcagattcacatcctg

SEQ ID NO: 26 - DNA, 28 bp, artificial sequence
  primer
gaatctcttcatggtgcctgatgacccc

SEQ ID NO: 27 - DNA, 28 bp, artificial sequence
  primer
gaatctcttcatggtgcctgatgacccc

SEQ ID NO: 28 - DNA, 23 bp, artificial sequence
  primer
atcatggaggctgaggcaccgca

SEQ ID NO: 29 - DNA, 34 bp, artificial sequence
  primer
caaagacagtatcatctgaagttgcctaataagg

SEQ ID NO: 30 - DNA, 34 bp, artificial sequence
  primer
atttcaattcaaagcattgtcttataaaaagcag

SEQ ID NO: 31 - DNA, 34 bp, artificial sequence
  primer
tcaagaactttatctgtgggagctgatttgcacc

SEQ ID NO: 32 - DNA, 34 bp, artificial sequence
  primer
ttttttcagaacagtgaacgatttaaagggagac

SEQ ID NO: 33 - DNA, 25 bp, artificial sequence
  primer
gttggattccatgaattggtattac

SEQ ID NO: 34 - DNA, 27 bp, artificial sequence
  primer
atttctctctaaagcttaaatgtgagg

SEQ ID NO: 35 - DNA, 25 bp, artificial sequence
  primer
actaaaacactgaaaaaaaatgccg

SEQ ID NO: 36 - DNA, 32 bp, artificial sequence
  primer
ttgccaatgcaaaatacagttgtcattaatcc

SEQ ID NO: 37 - DNA, 37 bp, artificial sequence
  primer
aacatctaccaaaatagttccctaaataggtttgtgc

SEQ ID NO: 38 - DNA, 18 bp, artificial sequence
  primer
tggccgtgaacctggtgg

SEQ ID NO: 39 - DNA, 23 bp, artificial sequence
  primer
cttccccagggaccaatcattac

SEQ ID NO: 40 - DNA, 23 bp, artificial sequence
  primer
ctgctcctacgtctgctggtttc

SEQ ID NO: 41 - DNA, 21 bp, artificial sequence
  primer
agctccccatcagcttccttg

SEQ ID NO: 42 - DNA, 23 bp, artificial sequence
  primer
cccttcccttttctccttcccta

SEQ ID NO: 43 - DNA, 23 bp, artificial sequence
  primer
actcagccttggggaagacaaag

SEQ ID NO: 44 - DNA, 19 bp, artificial sequence
  primer
ggaactaattccagagttc

SEQ ID NO: 45 - DNA, 19 bp, artificial sequence
  primer
ggcgaatacttaacttata

SEQ ID NO: 46 - DNA, 19 bp, artificial sequence
  primer
gtttcgctcaccatgcttc

SEQ ID NO: 47 - DNA, 19 bp, artificial sequence
  primer
aatggatgtaggaggactc

SEQ ID NO: 48 - DNA, 19 bp, artificial sequence
  primer
gaatgaggccatcgggagg

SEQ ID NO: 49 - DNA, 19 bp, artificial sequence
  primer
gctcttagcatggttcata

SEQ ID NO: 50 - DNA, 21 bp, artificial sequence
  primer
ccccaatgccagaagattcag

SEQ ID NO: 51 - DNA, 20 bp, artificial sequence
  primer
gtgggctcgtggaaggaaac

SEQ ID NO: 52 - DNA, 24 bp, artificial sequence
  primer
ctgccatcagcaaggaaatttttg

SEQ ID NO: 53 - DNA, 28 bp, artificial sequence
  primer
actcctccactttctttagagcttcacc

SEQ ID NO: 54 - DNA, 27 bp, artificial sequence
  primer
ggtctccagcgcctaagacacctccag

SEQ ID NO: 55 - DNA, 26 bp, artificial sequence
  primer
gggatctggggtcaggtgcttgcttg

SEQ ID NO: 56 - DNA, 22 bp, artificial sequence
  primer
gcggagaaagtgaacaacttcc

SEQ ID NO: 57 - DNA, 19 bp, artificial sequence
  primer
cttggctggctcacatctc

SEQ ID NO: 58 - DNA, 12 bp, artificial sequence
  primer
gactacggatcc

SEQ ID NO: 59 - DNA, 12 bp, artificial sequence
  primer
gatcaggaattc

SEQ ID NO: 60 - DNA, 12 bp, artificial sequence
  primer
gactacggatcc

SEQ ID NO: 61 - DNA, 12 bp, artificial sequence
  primer
gatcagctcgag

SEQ ID NO: 62 - DNA, 17 bp, Homo sapiens
  misc_feature (9)..(9): n is a, c, g, or t
ttgaaagantggccttc

SEQ ID NO: 63 - DNA, 17 bp, Homo sapiens
ttgaaagattggccttc

SEQ ID NO: 64 - DNA, 17 bp, Homo sapiens
  misc_feature (8)..(8): n is a, c, g, or t
  misc_feature (13)..(13): n is a, c, g, or t
  misc_feature (15)..(15): n is a, c, g, or t
tactcatnatgancncc

SEQ ID NO: 65 - DNA, 17 bp, Homo sapiens
  misc_feature (1)..(1): n is a, c, g, or t
  misc_feature (5)..(5): n is a, c, g, or t
nactnatcatgataacc

SEQ ID NO: 66 - DNA, 17 bp, Homo sapiens
  misc_feature (8)..(8): n is a, c, g, or t
  misc_feature (11)..(11): n is a, c, g, or t
  misc_feature (15)..(15): n is a, c, g, or t
tactcatnatnaccncc

SEQ ID NO: 67 - DNA, 17 bp, Homo sapiens
  misc_feature (1)..(1): n is a, c, g, or t
  misc_feature (5)..(6): n is a, c, g, or t
  misc_feature (8)..(8): n is a, c, g, or t
nactnntnatgataacc

SEQ ID NO: 68 - DNA, 18 bp, Homo sapiens
  misc_feature (10)..(10): n is a, c, g, or t
taatatggtngccggagg

SEQ ID NO: 69 - DNA, 18 bp, Homo sapiens
  misc_feature (10)..(10): n is a, c, g, or t
taatatggtngccggagg

SEQ ID NO: 70 - DNA, 16 bp, Homo sapiens
  misc_feature (8)..(8): n is a, c, g, or t
tgagtacngaaatcct

SEQ ID NO: 71 - DNA, 16 bp, Homo sapiens
tgagtacggaaatcct

SEQ ID NO: 72 - DNA, 12 bp, Homo sapiens
  misc_feature (6)..(6): n is a, c, g, or t
tgcccntggtct

SEQ ID NO: 73 - DNA, 12 bp, Homo sapiens
tgcccgtggtct